(19) United States
(12) Patent Application Publication
(10) Pub. No.: US 2015/0065362 A1
(43) Pub. Date: Mar. 5, 2015

(54) DYNAMIC METHODS FOR DIAGNOSIS AND PROGNOSIS OF CANCER

(71) Applicant: Atossa Genetics Inc., Seattle, WA (US)

(72) Inventors: Balázs Gyorffy, Budapest (HU); Steven C. Quay, Seattle, WA (US); Shu-Chih Chen, Seattle, WA (US)

(21) Appl. No.: 14/472,176

(22) Filed: Aug. 28, 2014

Related U.S. Application Data
(60) Provisional application No. 61/871,503, filed on Aug. 29, 2013.
(60) Provisional application No. 61/871,677, filed on Aug. 29, 2013.

(51) Int. Cl. G06F 19/00; G06F 19/22; C12Q 1/68

(52) U.S. Cl. CPC G06F 19/345; C12Q 1/6886; G06F 19/3431; G06F 19/22; C12Q 2600/158; C12Q 2600/112
USPC 506/8; 506/39

(57) ABSTRACT

Disclosed herein are computer-based systems, media and methods of generating dynamic classifiers and uses thereof. The dynamic classifiers may be generated from a subset of cases and/or a subset of genes that have a molecular similarity to a subject suffering from a cancer. Thus, the dynamic classifiers may be subject-specific. The dynamic classifiers may be used in the diagnosis, prognosis and/or monitoring of a status or outcome of a cancer in a subject in need thereof.

### CROSS-REFERENCE

This application claims the benefit of U.S. Provisional Application No. 61/871,503, filed Aug. 29, 2013, and U.S. Provisional Application No. 61/871,677 also filed Aug. 29, 2013, both of which applications are incorporated herein by reference in their entirety.

### BACKGROUND OF THE INVENTION

In the last decade, numerous multigene prognostic tests have been developed for breast cancer (S. Paik, S. Shak, G. Tang et al., N Engl J Med 351 (27), 2817 (2004); J. S. Parker, M. Mullins, M. C. Cheang et al., J Clin Oncol 27 (8), 1160 (2009); C. Sotiriou, P. Wirapati, S. Loi et al., J Natl Cancer Inst 98 (4), 262 (2006); L. J. van't Veer, H. Dai, M. J. van de Vijver et al., Nature 415 (6871), 530 (2002)). All of these assays have been developed from relatively small training sets and the informative genes were selected from molecularly heterogeneous populations. For example, the 70 genes included in MammaPrint were defined from a mixed cohort of 78 estrogen receptor (ER)-positive and -negative cases4. The 21 genes in OncotypeDX were derived from 233 ER positive, lymph node negative patients and the 97 genes of the Genomic Grade Index (GGI) were selected from 64 estrogen receptor positive tumors (S. Paik, S. Shak, G. Tang et al., N Engl J Med 351 (27), 2817 (2004); C. Sotiriou, P. Wirapati, S. Loi et al., J Natl Cancer Inst 98 (4), 262 (2006)). Although the prognostic performance of these assays have been validated in independent cases, their predictive performance may not be optimal due to the relatively small and heterogeneous training sets that were used for assay discovery (A. Rhodes, B. Jasani, A. J. Balaton et al., J Clin Pathol 53 (9), 688 (2000); A. Rhodes, B. Jasani, A. J. Balaton et al., Am J Clin Pathol 115 (1), 44 (2001); L. J. Layfield, N. Goldstein, K. R. Perkinson et al., Breast J 9 (3), 257 (2003)).

Twenty years after the development of the first gene expression arrays, several thousands of gene expression profiles with clinical annotation are now available from breast cancers. By using much larger and molecularly more homogeneous training sets, we developed a dynamic system which improved the accuracy of multi-gene prognostic signatures. In some instances, they dynamic system comprises a selection of the most molecularly similar cases to a test case from a large training case pool of cases to develop a unique, case-specific predictor which is applied to the test case. In some instances, the dynamic system defines a new training sub-cohort for each new test case and selects a new set of informative genes. In some instances, the dynamic classification process develops predictors built from a subset of cases with the greatest similarity to the test case.

### SUMMARY OF THE INVENTION

Described herein, in certain embodiments, are computer-implemented methods for generating dynamic classifiers. In some embodiments, the dynamic classifiers are case-specific. Additionally, in some instances, the dynamic classifiers are based on comparative analysis of a plurality of cancer cases to a cancer in a subject. In some embodiments, the method for generating a dynamic classifier comprises (a) receiving, by a computer, data input, the data pertaining to a plurality of cancer cases; and (b) generating, by the computer, a dynamic classifier, wherein the dynamic classifier is based on a comparison of the data pertaining to the plurality of cancer cases to data pertaining to a subject suffering from a cancer. In some embodiments, the dynamic classifier comprises a subset of the plurality of cancer cases. Alternatively, or additionally, the dynamic classifier comprises a subset of the data pertaining to the plurality of cancer cases. In some embodiments, the dynamic classifiers are used to provide a prognostic output. In other instances, the dynamic classifiers are used to provide a predictive output. In some embodiments, the cancer is a breast cancer.

Also described herein, in certain embodiments, are computer-implemented methods for diagnosing, predicting, or monitoring a status or outcome of a cancer in a subject in need thereof. Generally, the computer-implemented methods comprise (a) receiving, by a computer, data input, the data pertaining to a plurality of cancer cases; (b) generating, by the computer, a case-specific output, wherein the case-specific output comprises a subset of the plurality of cancer cases, a subset of the data pertaining to the plurality of cancer cases, or a combination thereof, and wherein the case-specific output is based on a comparison of the data pertaining to the plurality of cancer cases to data pertaining to a subject suffering from a cancer; and (c) generating, by the computer, a biomedical output, the biomedical output comprising a comparison of the data of the case-specific output to the data of the subject suffering from the cancer. In some embodiments, the method further comprises diagnosing, predicting or monitoring, by the computer, a status or outcome of the cancer in the subject based on the biomedical output. In some embodiments, the cancer is a breast cancer.

Also disclosed herein, in some embodiments, are dynamic computer-implemented systems for generating dynamic classifiers. In some instances, the system comprises (a) a digital processing device comprising an operating system configured to perform executable instructions and a memory device; and (b) a computer program including instructions executable by the digital processing device to create an application comprising: (i) a software module configured to receive data input, the data pertaining to a plurality of cancer cases; and (ii) a software module configured to generate a dynamic classifier. In some embodiments, the dynamic classifier comprises a subset of the plurality of cancer cases, a subset of the data pertaining to the plurality of cancer cases, or a combination thereof. In some embodiments, generating the dynamic classifier comprises comparing the data pertaining to the plurality of cancer cases to the data pertaining to a subject suffering from a cancer. In some embodiments, the system further comprises one or more additional software modules configured to generate a biomedical output. In some embodiments, the biomedical output comprises a comparison of the data of the dynamic classifier to the data of the subject suffering from the cancer. In some embodiments, the cancer is a breast cancer.

Further disclosed herein, in some embodiments, are dynamic computer-implemented systems for diagnosing, predicting, or monitoring a status or outcome of a cancer in a subject in thereof. In some instances, the system comprises (a) a digital processing device comprising an operating system configured to perform executable instructions and a memory device; and (b) a computer program including instructions executable by the digital processing device to create an application comprising: (i) a software module configured to receive data input, the data pertaining to a plurality of cancer cases; (ii) a software module configured to generate a case-specific output, wherein the case specific output comprises a subset of the plurality of cancer cases, a subset of the data pertaining to the plurality of cancer cases, or a combination thereof; and (iii) a software module configured to generate a biomedical output, the biomedical output comprising a comparison of the data of the case-specific output to the data of the subject suffering from the cancer. In some embodiments, the cancer is a breast cancer.

Also disclosed herein, in some embodiments, are non-transitory computer-readable storage media for use in generating a dynamic classifier. In some embodiments, the non-transitory computer-readable storage media is encoded with a computer program. In some embodiments, the computer program includes instructions executable by a processor to create an application for generating a dynamic classifier. In some embodiments, the storage media comprises (a) a database, in a computer memory, of a plurality of cancer cases; (b) a software module configured to receive data input, the data pertaining to a plurality of cancer cases; and (c) a software module configured to generate a dynamic classifier, wherein the dynamic classifier comprises a subset of the plurality of cancer cases, a subset of the data pertaining to the plurality of cancer cases, or a combination thereof. In some embodiments, the storage media comprises one or more additional software modules configured to generate a biomedical output, the biomedical output comprising a comparison of the data of the dynamic classifier to the data of the subject suffering from the cancer. In some embodiments, the cancer is a breast cancer.

Also disclosed herein, in some embodiments, are non-transitory computer-readable storage media for use in diagnosing, predicting or monitoring a status or outcome of a cancer in a subject in need thereof. In some embodiments, the non-transitory computer-readable storage media is encoded with a computer program. In some embodiments, the computer program includes instructions executable by a processor to create an application for diagnosing, predicting or monitoring a status or outcome of a cancer in a subject in need thereof. In some embodiments, the application comprises (a) a database, in a computer memory, of a plurality of cancer cases; (b) a software module configured to receive data input, the data pertaining to a plurality of cancer cases; (c) a software module configured to generate a case-specific output, wherein the case specific output comprises a subset of the plurality of cancer cases, a subset of the data pertaining to the plurality of cancer cases, or a combination thereof; and (d) a software module configured to generate a biomedical output, the biomedical output comprising a comparison of the data of the case-specific output to the data of the subject suffering from the cancer. In some embodiments, the cancer is a breast cancer.

In some embodiments, the systems, media and methods disclosed herein comprise data input. In some embodiments, the data input comprises one or more of: case identifiers, gene expression data, clinical survival information, survival annotation, treatment annotation, clinical information, stage of the cancer, ethnicity, age, age at diagnosis, age at death, gender, therapeutic regimen, response to a therapeutic regimen, efficacy of a therapeutic regimen, biopsy, clinical tumor staging, tumor pathological staging, lymph node status, or a combination thereof. In some embodiments, the data input comprises gene expression data. In some embodiments, the gene expression data comprises raw gene expression data.

In some embodiments, the data input is provided by upload of an output from one or more databases or data sources comprising cancer information. In some embodiments, the one or more databases or data sources are selected from medical records, clinical notes, genomic databases, biomedical databases, clinical trial databases, scientific databases, disease databases, oncogenic databases, biomarker databases, transcriptome databases, mutation databases, epigenomic databases, microbiome databases, or a combination thereof. In some embodiments, the one or more databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof. In some embodiments, the data input is provided by manual data entry. In some embodiments, the output from the one or more databases is in one or more formats selected from: a database, a spreadsheet, comma-separated values, tab-separated values, or a combination thereof.

In some embodiments, the systems, media and/or methods further comprise one or more additional software modules configured to rank two or more cancer cases of the plurality of cancer cases. In some embodiments, ranking comprises comparing data of the two or more cancer cases to data of the subject. In some embodiments, comparing the data of the two or more cancer cases to the data of the subject comprises comparing an expression profile of one or more genes of the two or more cancer cases to an expression profile of one or more genes of the subject. In some embodiments, comparing comprises determining the similarity of the two or more cancer cases to the subject. In some embodiments, determining the similarity of the two or more cancer cases to the subject comprises producing a global similarity matrix over a plurality of genes of the two or more cancer cases to a plurality of genes of the subject. In some embodiments, producing the global similarity matrix comprises computing Euclidean distance. In some embodiments, ranking comprises determining molecular similarity of the data of the two or more ranked cancer cases to the data of the subject.

In some embodiments, the systems, media and/or methods further comprise one or more additional software modules configured to generate a case-specific training subset based on the ranking of the two or more cancer cases. In some embodiments, the case-specific training subset comprises a subset of the plurality of cancer cases. In some embodiments, the subset of the plurality of cancer cases comprises the most similar cancer cases to the subject. In some embodiments, the subset of the plurality of breast cancer comprises at least two of the highest ranked cancer cases of the two or more ranked cancer cases. In some embodiments, the case-specific output comprises the case-specific training subset.

In some embodiments, the systems, media and/or methods further comprise one or more additional software modules configured to rank two or more genes of one or more cancer cases of the case-specific training subset. In some embodiments, ranking comprises comparing an expression level of the two or more genes of the one or more cancer cases to an expression level of two or more genes of the subject. In some embodiments, ranking comprises performing a Kaplan-Meier survival analysis for two or more genes of the one or more cancer cases of the case-specific training subset. In some embodiments, ranking is based on one or more of: p-value, hazard ratio, or a combination thereof.

In some embodiments, the systems, media and methods further comprise one or more additional software modules configured to generate a case-specific gene set based on the ranking of the two or more genes. In some embodiments, the case-specific gene set comprises the subset of the data pertaining to the plurality of cancer cases. In some embodiments, the subset of the data comprises one or more of the highest ranked genes. In some embodiments, the case-specific output comprises the case-specific gene set. In some embodiments, the case-specific output is in one or more formats selected from: a database, a spreadsheet, comma-separated values, tab-separated values, or a combination thereof.

In some embodiments, the systems, media and/or methods comprise one or more biomedical outputs. In some embodiments, the biomedical output comprises one or more molecular classifications. In some embodiments, the one or more molecular classifications are based on a comparison of an average expression level of the one or more highest ranked genes of the case-specific output to an average expression level of one or more genes of the subject. In some embodiments, the biomedical output further comprises one or more training set assessments. In some embodiments, the one or more training set assessments are based on a comparison of the case-specific output to one or more additional subjects suffering from a cancer. In some embodiments, the comparison of the case specific output to the one or more additional subjects is based on Kaplan-Meier analysis.

In some embodiments, the systems, media and/or methods further comprise one or more dynamic classifiers. In some embodiments, the dynamic classifiers are based on a comparison of data input from a plurality of cancer cases to data input from a subject suffering from a cancer. In some embodiments, the dynamic classifiers are based on a comparison of data from one or more case-specific outputs to data from a subject suffering from a cancer. In some embodiments, the dynamic classifiers are based on a comparison of data from one or more biomedical outputs to data from a subject suffering from a cancer. In some embodiments, the dynamic classifiers comprise a subset of cancer cases from the plurality of cancer cases. In some embodiments, the dynamic classifiers comprise a subset of cancer cases from the case-specific output. In some embodiments, the dynamic classifiers comprise a subset of cancer cases from the biomedical output. In some embodiments, the dynamic classifiers comprise a subset of cancer cases that are a molecular match to a cancer from a subject. In some embodiments, the dynamic classifiers comprise a subset of genes from the plurality of cancer cases. In some embodiments, the dynamic classifiers comprise a subset of genes from the case-specific output. In some embodiments, the dynamic classifiers comprise a subset of genes from the biomedical output. In some embodiments, the dynamic classifiers comprise a subset of genes that are a molecular match to a cancer from a subject.

In some embodiments, the systems, media and/or methods further comprise one or more additional software modules configured to diagnose, predict, or monitor a status or outcome of the cancer in the subject. In some embodiments, diagnosing, predicting or monitoring the status or outcome comprises a prognostic output. In some embodiments, the prognostic output comprises a likelihood of recurrence of the cancer in the subject. In some embodiments, the prognostic output comprises a likelihood of lymph node invasion. In some embodiments, the likelihood of lymph node invasion is at the time of diagnosis. In some embodiments, the prognostic output comprises a likelihood of metastasis of the cancer in the subject. In some embodiments, diagnosing, predicting or monitoring the status or outcome comprises a predictive output. In some embodiments, diagnosing, predicting, or monitoring the status or outcome is based on the biomedical output comprising one or more molecular classifications and one or more training set assessments. In some embodiments, diagnosing, predicting, or monitoring the status or outcome is based on comparing the similarity of the one or more molecular classifications and the one or more training set assessments. In some embodiments, diagnosing, predicting, or monitoring the status or outcome is definitive when the one or more molecular classifications are similar to the one or more training set assessments. In some embodiments, diagnosing, predicting, or monitoring the status or outcome is indefinite when the one or more molecular classifications contradict the one or more training set assessments. In some embodiments, diagnosing, predicting, or monitoring the status or outcome is indefinite when the one or more molecular classifications are not significant. In some embodiments, diagnosing, predicting, or monitoring further comprises generating one or more biomedical reports. In some embodiments, the one or more biomedical reports comprise information pertaining to the diagnosis, prediction, or monitoring of the status or outcome of the cancer in the subject.

In some embodiments, the systems, media and/or methods further comprise one or more additional software modules configured to transmit the case-specific output, biomedical output, biomedical report, dynamic classifier or a combination thereof. In some embodiments, the case-specific output, biomedical output, biomedical report and/or dynamic classifier are transmitted via a web application. In some embodiments, the web application is implemented as software-as-a-service.

In some embodiments, the systems, media and/or methods further comprise one or more additional software modules configured to add comparator data. In some embodiments, the comparator data comprises a static predictor. In some embodiments, the static predictor is user-selectable. In some embodiments, the static predictor is selected from the group comprising a 21-gene recurrence score, 70-gene Mammaprint signature classifier, and 97-gene genomic grade index (GGI). In some embodiments, the system further comprises one or more additional software modules configured to compare the biomedical output to one or more static outputs, wherein the static outputs are based on one or more static predictors. In some embodiments, the system further comprises one or more additional software modules configured to compare the dynamic classifier to one or more static outputs, wherein the static outputs are based on one or more static predictors. In some embodiments, the dynamic classifier outperforms one or more static predictors. In some embodiments, a performance of the dynamic classifier is based on accuracy, sensitivity, specificity or a combination thereof. In some embodiments, the dynamic classifier outperforms the one or more static predictors when the accuracy, sensitivity and/or specificity of the dynamic classifier is greater than the accuracy, sensitivity and/or specificity of the one or more static predictors.

Disclosed herein are dynamic computer-implemented methods for generating one or more dynamic classifiers. In some embodiments, the method comprises (a) receiving, by a computer, data input, the data pertaining to a plurality of cancer cases; and (b) generating, by the computer, a dynamic classifier, wherein the dynamic classifier is based on a comparison of the data pertaining to the plurality of breast cancer cases to data pertaining to a subject suffering from a breast cancer. In some embodiments, the dynamic classifier comprises a subset of the plurality of breast cancer cases. Alternatively, or additionally, the dynamic classifier comprises a subset of the data pertaining to the plurality of breast cancer cases. In some embodiments, the dynamic classifiers are used to provide a prognostic output. In other instances, the dynamic classifiers are used to provide a predictive output. In some embodiments, the data input comprises one or more of: case identifiers, gene expression data, clinical survival information, survival annotation, treatment annotation, clinical information, stage of the breast cancer, ethnicity, age, age at diagnosis, age at death, gender, therapeutic regimen, response to a therapeutic regimen, efficacy of a therapeutic regimen, biopsy, clinical tumor staging, tumor pathological staging, lymph node status, or a combination thereof. In some embodiments, the data input comprises gene expression data. In some embodiments, the gene expression data comprises raw gene expression data. In some embodiments, the gene expression data comprises unprocessed gene expression data. In some embodiments, the gene expression data is generated on one or more arrays. In some embodiments, the one or more arrays comprise HG-U133A (GPL6) or HG-U133 Plus 2.0 (GPL570) arrays. In some embodiments, the data input is provided by upload of an output from one or more databases or data sources comprising breast cancer information. In some embodiments, the one or more databases or data sources are selected from medical records, clinical notes, genomic databases, biomedical databases, clinical trial databases, scientific databases, disease databases, oncogenic databases, biomarker databases, transcriptome databases, mutation databases, epigenomic databases, microbiome databases, or a combination thereof. In some embodiments, the one or more databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof. In some embodiments, the data input is provided by manual data entry. In some embodiments, the output from the one or more databases is in one or more formats selected from: a database, a spreadsheet, comma-separated values, tab-separated values, or a combination thereof. In some embodiments, the method further comprises ranking two or more breast cancer cases of the plurality of breast cancer cases. In some embodiments, ranking comprises comparing data of the two or more breast cancer cases to data of the subject. In some embodiments, comparing the data of the two or more breast cancer cases to the data of the subject comprises comparing an expression profile of one or more genes of the two or more breast cancer cases to an expression profile of one or more genes of the subject. In some embodiments, comparing further comprises determining the similarity of the two or more breast cancer cases to the subject. In some embodiments, determining the similarity of the two or more breast cancer cases to the subject comprises producing a global similarity matrix over a plurality of genes of the two or more breast cancer cases to a plurality of genes of the subject. In some embodiments, producing the global similarity matrix comprises computing Euclidean distance. In some embodiments, ranking comprises determining molecular similarity of the data of the two or more ranked breast cancer cases to the data of the subject. In some embodiments, the method further comprises producing a case-specific training subset based on the ranking of the two or more breast cancer cases. In some embodiments, the case-specific training subset comprises a subset of the plurality of breast cancer cases. In some embodiments, the subset of the plurality of breast cancer cases comprises the most similar breast cancer cases to the subject. In some embodiments, the subset of the plurality of breast cancer comprises at least two of the highest ranked breast cancer cases of the two or more ranked breast cancer cases. In some embodiments, the case-specific output comprises the case-specific training subset. In some embodiments, the method further comprises ranking two or more genes of one or more breast cancer cases of the case-specific training subset. In some embodiments, ranking comprises comparing an expression level of the two or more genes of the one or more breast cancer cases to an expression level of two or more genes of the subject. In some embodiments, ranking comprises performing a Kaplan-Meier survival analysis for two or more genes of the one or more breast cancer cases of the case-specific training subset. In some embodiments, ranking is based on one or more of: p-value, hazard ratio, or a combination thereof. In some embodiments, the method further comprises producing a case-specific gene set based on the ranking of the two or more genes. In some embodiments, the case-specific gene set comprises the subset of the data pertaining to the plurality of breast cancer cases. In some embodiments, the subset of the data comprises one or more of the highest ranked genes. In some embodiments, the case-specific output comprises the case-specific gene set. In some embodiments, the case-specific output is in one or more formats selected from: a database, a spreadsheet, comma-separated values, tab-separated values, or a combination thereof. In some embodiments, the biomedical output comprises one or more molecular classifications. In some embodiments, the one or more molecular classifications are based on a comparison of an average expression level of the one or more highest ranked genes of the case-specific output to an average expression level of one or more genes of the subject. In some embodiments, the biomedical output further comprises one or more training set assessments. In some embodiments, the one or more training set assessments are based on a comparison of the case-specific output to one or more additional subjects suffering from a breast cancer. In some embodiments, the comparison of the case specific output to the one or more additional subjects is based on Kaplan-Meier analysis. In some embodiments, diagnosing, predicting or monitoring the status or outcome comprises a prognostic output. In some embodiments, the prognostic output comprises a likelihood of recurrence of the breast cancer in the subject. In some embodiments, the prognostic output comprises a likelihood of lymph node invasion. In some embodiments, the likelihood of lymph node invasion is at the time of diagnosis. In some embodiments, the prognostic output comprises a likelihood of metastasis of the breast cancer in the subject. In some embodiments, diagnosing, predicting or monitoring the status or outcome comprises a predictive output. In some embodiments, the predictive output comprises predicting a response of the subject to a therapeutic regimen. In some embodiments, the therapeutic regimen comprises a chemotherapeutic agent. In some embodiments, diagnosing, predicting or monitoring the status or outcome comprises determining a stage of the breast cancer in the subject. In some embodiments, diagnosing, predicting or monitoring the status or outcome comprises treating the breast cancer in the subject. In some embodiments, diagnosing, predicting or monitoring the status or outcome comprises determining, modifying, or maintaining a therapeutic regimen. In some embodiments, diagnosing, predicting or monitoring the status or outcome comprises administering a therapeutic regimen. In some embodiments, diagnosing, predicting, or monitoring the status or outcome is based on the biomedical output comprising the one or more molecular classifications and one or more training set assessments. In some embodiments, diagnosing, predicting, or monitoring the status or outcome is based on comparing the similarity of the one or more molecular classifications and the one or more training set assessments. In some embodiments, diagnosing, predicting, or monitoring the status or outcome is definitive when the one or more molecular classifications are similar to the one or more training set assessments. In some embodiments, diagnosing, predicting, or monitoring the status or outcome is indefinite when the one or more molecular classifications contradict the one or more training set assessments. In some embodiments, diagnosing, predicting, or monitoring the status or outcome is indefinite when the one or more molecular classifications are not significant. In some embodiments, diagnosing, predicting, or monitoring further comprises generating one or more biomedical reports. In some embodiments, the one or more biomedical reports comprise information pertaining to the diagnosis, prediction, or monitoring of the status or outcome of the cancer in the subject. In some embodiments, the method further comprises transmitting the case-specific output, biomedical output, biomedical report, dynamic classifier or a combination thereof. In some embodiments, the case-specific output, biomedical output, and/or biomedical report are transmitted via a web application. In some embodiments, the web application is implemented as software-as-a-service. In some embodiments, the case-specific output, biomedical output, biomedical report and/or dynamic classifier are transmitted to one or more users. In some embodiments, the one or more users are one or more subjects suffering from a cancer, doctors, nurses, physician's assistants, hospital personnel, medical personnel, medical consultants, medical counselors, health advisors, medical experts, researchers, analysts, or a combination thereof. In some embodiments, the method further comprises comparing the biomedical output to one or more static outputs, wherein the static outputs are based one or more static predictors. In some embodiments, the one or more static predictors comprise a 21-gene recurrence score, 70-gene Mammaprint signature classifier, 97-gene genomic grade index (GGI), or a combination thereof. In some embodiments, the one or more static predictors are user-selectable.

Disclosed herein are dynamic computer-implemented methods for diagnosing, predicting or monitoring a status or outcome of a breast cancer in a subject in need thereof. In some embodiments, the method comprises (a) receiving, by a computer, data input, the data pertaining to a plurality of breast cancer cases; (b) generating, by the computer, a case-specific output, wherein the case-specific output comprises a subset of the plurality of breast cancer cases, a subset of the data pertaining to the plurality of breast cancer cases, or a combination thereof, and wherein the case-specific output is based on a comparison of the data pertaining to the plurality of breast cancer cases to data pertaining to a subject suffering from a breast cancer; (c) generating, by the computer, a biomedical output, the biomedical output comprising a comparison of the data of the case-specific output to the data of the subject suffering from the breast cancer; and (d) diagnosing, predicting or monitoring, by the computer, a status or outcome of the breast cancer in the subject based on the biomedical output. In some embodiments, the data input comprises one or more of: case identifiers, gene expression data, clinical survival information, survival annotation, treatment annotation, clinical information, stage of the breast cancer, ethnicity, age, age at diagnosis, age at death, gender, therapeutic regimen, response to a therapeutic regimen, efficacy of a therapeutic regimen, biopsy, clinical tumor staging, tumor pathological staging, lymph node status, or a combination thereof. In some embodiments, the data input comprises gene expression data. In some embodiments, the gene expression data comprises raw gene expression data. In some embodiments, the gene expression data comprises unprocessed gene expression data. In some embodiments, the gene expression data is generated on one or more arrays. In some embodiments, the one or more arrays comprise HG-U133A (GPL6) or HG-U133 Plus 2.0 (GPL570) arrays. In some embodiments, the data input is provided by upload of an output from one or more databases or data sources comprising breast cancer information. In some embodiments, the one or more databases or data sources are selected from a medical records, clinical notes, genomic databases, biomedical databases, clinical trial databases, scientific databases, disease databases, oncogenic databases, biomarker databases, transcriptome databases, mutation databases, epigenomic databases, microbiome databases or a combination thereof. In some embodiments, the one or more databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof. In some embodiments, the data input is provided by manual data entry. In some embodiments, the output from the one or more databases is in one or more formats selected from: a database, a spreadsheet, comma-separated values, tab-separated values, or a combination thereof. In some embodiments, the method further comprises ranking two or more breast cancer cases of the plurality of breast cancer cases. In some embodiments, ranking comprises comparing data of the two or more breast cancer cases to data of the subject. In some embodiments, comparing the data of the two or more breast cancer cases to the data of the subject comprises comparing an expression profile of one or more genes of the two or more breast cancer cases to an expression profile of one or more genes of the subject. In some embodiments, comparing further comprises determining the similarity of the two or more breast cancer cases to the subject. In some embodiments, determining the similarity of the two or more breast cancer cases to the subject comprises producing a global similarity matrix over a plurality of genes of the two or more breast cancer cases to a plurality of genes of the subject. In some embodiments, producing the global similarity matrix comprises computing Euclidean distance. In some embodiments, ranking comprises determining molecular similarity of the data of the two or more ranked breast cancer cases to the data of the subject. In some embodiments, the method further comprises producing a case-specific training subset based on the ranking of the two or more breast cancer cases. In some embodiments, the case-specific training subset comprises a subset of the plurality of breast cancer cases. In some embodiments, the subset of the plurality of breast cancer cases comprises the most similar breast cancer cases to the subject. In some embodiments, the subset of the plurality of breast cancer comprises at least two of the highest ranked breast cancer cases of the two or more ranked breast cancer cases. In some embodiments, the case-specific output comprises the case-specific training subset. In some embodiments, the method further comprises ranking two or more genes of one or more breast cancer cases of the case-specific training subset. In some embodiments, ranking comprises comparing an expression level of the two or more genes of the one or more breast cancer cases to an expression level of two or more genes of the subject. In some embodiments, ranking comprises performing a Kaplan-Meier survival analysis for two or more genes of the one or more breast cancer cases of the case-specific training subset. In some embodiments, ranking is based on one or more of: p-value, hazard ratio, or a combination thereof. In some embodiments, the method further comprises producing a case-specific gene set based on the ranking of the two or more genes. In some embodiments, the case-specific gene set comprises the subset of the data pertaining to the plurality of breast cancer cases. In some embodiments, the subset of the data comprises one or more of the highest ranked genes. In some embodiments, the case-specific output comprises the case-specific gene set. In some embodiments, the case-specific output is in one or more formats selected from: a database, a spreadsheet, comma-separated values, tab-separated values, or a combination thereof. In some embodiments, the biomedical output comprises one or more molecular classifications. In some embodiments, the one or more molecular classifications are based on a comparison of an average expression level of the one or more highest ranked genes of the case-specific output to an average expression level of one or more genes of the subject. In some embodiments, the biomedical output further comprises one or more training set assessments. In some embodiments, the one or more training set assessments are based on a comparison of the case-specific output to one or more additional subjects suffering from a breast cancer. In some embodiments, the comparison of the case specific output to the one or more additional subjects is based on Kaplan-Meier analysis. In some embodiments, diagnosing, predicting or monitoring the status or outcome comprises a prognostic output. In some embodiments, the prognostic output comprises a likelihood of recurrence of the breast cancer in the subject. In some embodiments, the prognostic output comprises a likelihood of lymph node invasion. In some embodiments, the likelihood of lymph node invasion is at the time of diagnosis. In some embodiments, the prognostic output comprises a likelihood of metastasis of the breast cancer in the subject. In some embodiments, diagnosing, predicting or monitoring the status or outcome comprises a predictive output. In some embodiments, the predictive output comprises predicting a response of the subject to a therapeutic regimen. In some embodiments, diagnosing, predicting or monitoring the status or outcome comprises determining a stage of the breast cancer in the subject. In some embodiments, diagnosing, predicting or monitoring the status or outcome comprises treating the breast cancer in the subject. In some embodiments, diagnosing, predicting or monitoring the status or outcome comprises determining, modifying, or maintaining a therapeutic regimen. In some embodiments, diagnosing, predicting or monitoring the status or outcome comprises administering a therapeutic regimen. In some embodiments, diagnosing, predicting, or monitoring the status or outcome is based on the biomedical output comprising the one or more molecular classifications and one or more training set assessments. In some embodiments, diagnosing, predicting, or monitoring the status or outcome is based on comparing the similarity of the one or more molecular classifications and the one or more training set assessments. In some embodiments, diagnosing, predicting, or monitoring the status or outcome is definitive when the one or more molecular classifications are similar to the one or more training set assessments. In some embodiments, diagnosing, predicting, or monitoring the status or outcome is indefinite when the one or more molecular classifications contradict the one or more training set assessments. In some embodiments, diagnosing, predicting, or monitoring the status or outcome is indefinite when the one or more molecular classifications are not significant. In some embodiments, diagnosing, predicting, or monitoring further comprises generating one or more biomedical reports. In some embodiments, the one or more biomedical reports comprise information pertaining to the diagnosis, prediction, or monitoring of the status or outcome of the cancer in the subject. In some embodiments, the method further comprises transmitting the case-specific output, biomedical output, biomedical report, dynamic classifier or a combination thereof. In some embodiments, the case-specific output, biomedical output, and/or biomedical report are transmitted via a web application. In some embodiments, the web application is implemented as software-as-a-service. In some embodiments, the case-specific output, biomedical output, biomedical report and/or dynamic classifier are transmitted to one or more users. In some embodiments, the one or more users are one or more subjects suffering from a cancer, doctors, nurses, physician's assistants, hospital personnel, medical personnel, medical consultants, medical counselors, health advisors, medical experts, researchers, analysts, or a combination thereof. In some embodiments, the method further comprises comparing the biomedical output to one or more static outputs, wherein the static outputs are based one or more static predictors. In some embodiments, the one or more static predictors comprise a 21-gene recurrence score, 70-gene Mammaprint signature classifier, 97-gene genomic grade index (GGI), or a combination thereof. In some embodiments, the one or more static predictors are user-selectable.

Also disclosed herein, in some embodiments, are dynamic computer-implemented systems for generating dynamic classifiers. In some instances, the system comprises (a) a digital processing device comprising an operating system configured to perform executable instructions and a memory device; and (b) a computer program including instructions executable by the digital processing device to create an application comprising: (i) a software module configured to receive data input, the data pertaining to a plurality of breast cancer cases; and (ii) a software module configured to generate a dynamic classifier. In some embodiments, the dynamic classifier comprises a subset of the plurality of breast cancer cases, a subset of the data pertaining to the plurality of breast cancer cases, or a combination thereof. In some embodiments, generating the dynamic classifier comprises comparing the data pertaining to the plurality of breast cancer cases to the data pertaining to a subject suffering from a breast cancer. In some embodiments, the system further comprises one or more additional software modules configured to generate a biomedical output. In some embodiments, the biomedical output comprises a comparison of the data of the dynamic classifier to the data of the subject suffering from the breast cancer. In some embodiments, the data input comprises one or more of: case identifiers, gene expression data, clinical survival information, survival annotation, treatment annotation, clinical information, stage of the breast cancer, ethnicity, age, age at diagnosis, age at death, gender, therapeutic regimen, response to a therapeutic regimen, efficacy of a therapeutic regimen, biopsy, clinical tumor staging, tumor pathological staging, lymph node status, or a combination thereof. In some embodiments, the data input comprises gene expression data. In some embodiments, the gene expression data comprises raw gene expression data. In some embodiments, the data input is provided by upload of an output from one or more databases or data sources comprising breast cancer information. In some embodiments, the one or more databases or data sources are selected from medical records, clinical notes, genomic databases, biomedical databases, clinical trial databases, scientific databases, disease databases, oncogenic databases, biomarker databases, transcriptome databases, mutation databases, epigenomic databases, microbiome databases, or a combination thereof. In some embodiments, the one or more databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof. In some embodiments, the data input is provided by manual data entry. In some embodiments, the output from the one or more databases is in one or more formats selected from: a database, a spreadsheet, comma-separated values, tab-separated values, or a combination thereof. In some embodiments, the system further comprises one or more additional software modules configured to rank two or more breast cancer cases of the plurality of breast cancer cases. In some embodiments, ranking comprises comparing data of the two or more breast cancer cases to data of the subject. In some embodiments, comparing the data of the two or more breast cancer cases to the data of the subject comprises comparing an expression profile of one or more genes of the two or more breast cancer cases to an expression profile of one or more genes of the subject. In some embodiments, comparing comprises determining the similarity of the two or more breast cancer cases to the subject. In some embodiments, determining the similarity of the two or more breast cancer cases to the subject comprises producing a global similarity matrix over a plurality of genes of the two or more breast cancer cases to a plurality of genes of the subject. In some embodiments, producing the global similarity matrix comprises computing Euclidean distance. In some embodiments, ranking comprises determining molecular similarity of the data of the two or more ranked breast cancer cases to the data of the subject. In some embodiments, the system further comprises one or more additional software modules configured to generate a case-specific training subset based on the ranking of the two or more breast cancer cases. In some embodiments, the case-specific training subset comprises a subset of the plurality of breast cancer cases. In some embodiments, the subset of the plurality of breast cancer cases comprises the most similar breast cancer cases to the subject. In some embodiments, the subset of the plurality of breast cancer comprises at least two of the highest ranked breast cancer cases of the two or more ranked breast cancer cases. In some embodiments, the case-specific output comprises the case-specific training subset. In some embodiments, the system further comprises one or more additional software modules configured to rank two or more genes of one or more breast cancer cases of the case-specific training subset. In some embodiments, ranking comprises comparing an expression level of the two or more genes of the one or more breast cancer cases to an expression level of two or more genes of the subject. In some embodiments, ranking comprises performing a Kaplan-Meier survival analysis for two or more genes of the one or more breast cancer cases of the case-specific training subset. In some embodiments, ranking is based on one or more of: p-value, hazard ratio, or a combination thereof. In some embodiments, the system further comprises one or more additional software modules configured to generate a case-specific gene set based on the ranking of the two or more genes. In some embodiments, the case-specific gene set comprises the subset of the data pertaining to the plurality of breast cancer cases. In some embodiments, the subset of the data comprises one or more of the highest ranked genes. In some embodiments, the case-specific output comprises the case-specific gene set. In some embodiments, the case-specific output is in one or more formats selected from: a database, a spreadsheet, comma-separated values, tab-separated values, or a combination thereof. In some embodiments, the biomedical output comprises one or more molecular classifications. In some embodiments, the one or more molecular classifications are based on a comparison of an average expression level of the one or more highest ranked genes of the case-specific output to an average expression level of one or more genes of the subject. In some embodiments, the biomedical output further comprises one or more training set assessments. In some embodiments, the one or more training set assessments are based on a comparison of the case-specific output to one or more additional subjects suffering from a breast cancer. In some embodiments, the comparison of the case specific output to the one or more additional subjects is based on Kaplan-Meier analysis. In some embodiments, the system further comprises one or more additional software modules configured to diagnose, predict, or monitor a status or outcome of the breast cancer in the subject. In some embodiments, diagnosing, predicting or monitoring the status or outcome comprises a prognostic output. In some embodiments, the prognostic output comprises a likelihood of recurrence of the breast cancer in the subject. In some embodiments, the prognostic output comprises a likelihood of lymph node invasion. In some embodiments, the likelihood of lymph node invasion is at the time of diagnosis. In some embodiments, the prognostic output comprises a likelihood of metastasis of the breast cancer in the subject. In some embodiments, diagnosing, predicting or monitoring the status or outcome comprises a predictive output. In some embodiments, diagnosing, predicting, or monitoring the status or outcome is based on the biomedical output comprising one or more molecular classifications and one or more training set assessments. In some embodiments, diagnosing, predicting, or monitoring the status or outcome is based on comparing the similarity of the one or more molecular classifications and the one or more training set assessments. In some embodiments, diagnosing, predicting, or monitoring the status or outcome is definitive when the one or more molecular classifications are similar to the one or more training set assessments. In some embodiments, diagnosing, predicting, or monitoring the status or outcome is indefinite when the one or more molecular classifications contradict the one or more training set assessments. In some embodiments, diagnosing, predicting, or monitoring the status or outcome is indefinite when the one or more molecular classifications are not significant. In some embodiments, diagnosing, predicting, or monitoring further comprises generating one or more biomedical reports. In some embodiments, the one or more biomedical reports comprise information pertaining to the diagnosis, prediction, or monitoring of the status or outcome of the cancer in the subject. In some embodiments, the system further comprises one or more additional software modules configured to transmit the case-specific output, biomedical output, biomedical report, dynamic classifier or a combination thereof. In some embodiments, the case-specific output, biomedical output, biomedical report and/or dynamic classifier are transmitted via a web application. In some embodiments, the web application is implemented as software-as-a-service. In some embodiments, the system further comprises one or more additional software modules configured to add comparator data. In some embodiments, the comparator data comprises a static predictor. In some embodiments, the static predictor is user-selectable. In some embodiments, the static predictor is selected from the group comprising a 21-gene recurrence score, 70-gene Mammaprint signature classifier, and 97-gene genomic grade index (GGI). In some embodiments, the system further comprises one or more additional software modules configured to compare the biomedical output to one or more static outputs, wherein the static outputs are based on one or more static predictors. In some embodiments, the system further comprises one or more additional software modules configured to compare the dynamic classifier to one or more static outputs, wherein the static outputs are based on one or more static predictors.

Also disclosed herein are dynamic computer-implemented systems for generating one or more biomedical outputs. In some embodiments, the system comprises (a) a digital processing device comprising an operating system configured to perform executable instructions and a memory device; and (b) a computer program including instructions executable by the digital processing device to create an application comprising: (i) a software module configured to receive data input, the data pertaining to a plurality of breast cancer cases; (ii) a software module configured to generate a case-specific output, wherein the case specific output comprises a subset of the plurality of breast cancer cases, a subset of the data pertaining to the plurality of breast cancer cases, or a combination thereof; and (iii) a software module configured to generate a biomedical output, the biomedical output comprising a comparison of the data of the case-specific output to the data of the subject suffering from the breast cancer. In some embodiments, the data input comprises one or more of: case identifiers, gene expression data, clinical survival information, survival annotation, treatment annotation, clinical information, stage of the breast cancer, ethnicity, age, age at diagnosis, age at death, gender, therapeutic regimen, response to a therapeutic regimen, efficacy of a therapeutic regimen, biopsy, clinical tumor staging, tumor pathological staging, lymph node status, or a combination thereof. In some embodiments, the data input comprises gene expression data. In some embodiments, the gene expression data comprises raw gene expression data. In some embodiments, the data input is provided by upload of an output from one or more databases or data sources comprising breast cancer information. In some embodiments, the one or more databases or data sources are selected from medical records, clinical notes, genomic databases, biomedical databases, clinical trial databases, scientific databases, disease databases, oncogenic databases, biomarker databases, transcriptome databases, mutation databases, epigenomic databases, microbiome databases, or a combination thereof. In some embodiments, the one or more databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof. In some embodiments, the data input is provided by manual data entry. In some embodiments, the output from the one or more databases is in one or more formats selected from: a database, a spreadsheet, comma-separated values, tab-separated values, or a combination thereof. In some embodiments, the system further comprises one or more additional software modules configured to rank two or more breast cancer cases of the plurality of breast cancer cases. In some embodiments, ranking comprises comparing data of the two or more breast cancer cases to data of the subject. In some embodiments, comparing the data of the two or more breast cancer cases to the data of the subject comprises comparing an expression profile of one or more genes of the two or more breast cancer cases to an expression profile of one or more genes of the subject. In some embodiments, comparing comprises determining the similarity of the two or more breast cancer cases to the subject. In some embodiments, determining the similarity of the two or more breast cancer cases to the subject comprises producing a global similarity matrix over a plurality of genes of the two or more breast cancer cases to a plurality of genes of the subject. In some embodiments, producing the global similarity matrix comprises computing Euclidean distance. In some embodiments, ranking comprises determining molecular similarity of the data of the two or more ranked breast cancer cases to the data of the subject. In some embodiments, the system further comprises one or more additional software modules configured to generate a case-specific training subset based on the ranking of the two or more breast cancer cases. In some embodiments, the case-specific training subset comprises a subset of the plurality of breast cancer cases. In some embodiments, the subset of the plurality of breast cancer cases comprises the most similar breast cancer cases to the subject. In some embodiments, the subset of the plurality of breast cancer comprises at least two of the highest ranked breast cancer cases of the two or more ranked breast cancer cases. In some embodiments, the case-specific output comprises the case-specific training subset. In some embodiments, the system further comprises one or more additional software modules configured to rank two or more genes of one or more breast cancer cases of the case-specific training subset. In some embodiments, ranking comprises comparing an expression level of the two or more genes of the one or more breast cancer cases to an expression level of two or more genes of the subject. In some embodiments, ranking comprises performing a Kaplan-Meier survival analysis for two or more genes of the one or more breast cancer cases of the case-specific training subset. In some embodiments, ranking is based on one or more of: p-value, hazard ratio, or a combination thereof. In some embodiments, the system further comprises one or more additional software modules configured to generate a case-specific gene set based on the ranking of the two or more genes. In some embodiments, the case-specific gene set comprises the subset of the data pertaining to the plurality of breast cancer cases. In some embodiments, the subset of the data comprises one or more of the highest ranked genes. In some embodiments, the case-specific output comprises the case-specific gene set. In some embodiments, the case-specific output is in one or more formats selected from: a database, a spreadsheet, comma-separated values, tab-separated values, or a combination thereof. In some embodiments, the biomedical output comprises one or more molecular classifications. In some embodiments, the one or more molecular classifications are based on a comparison of an average expression level of the one or more highest ranked genes of the case-specific output to an average expression level of one or more genes of the subject. In some embodiments, the biomedical output further comprises one or more training set assessments. In some embodiments, the one or more training set assessments are based on a comparison of the case-specific output to one or more additional subjects suffering from a breast cancer. In some embodiments, the comparison of the case specific output to the one or more additional subjects is based on Kaplan-Meier analysis. In some embodiments, the system further comprises one or more additional software modules configured to diagnose, predict, or monitor a status or outcome of the breast cancer in the subject. In some embodiments, diagnosing, predicting or monitoring the status or outcome comprises a prognostic output. In some embodiments, the prognostic output comprises a likelihood of recurrence of the breast cancer in the subject. In some embodiments, the prognostic output comprises a likelihood of lymph node invasion. In some embodiments, the likelihood of lymph node invasion is at the time of diagnosis. In some embodiments, the prognostic output comprises a likelihood of metastasis of the breast cancer in the subject. In some embodiments, diagnosing, predicting or monitoring the status or outcome comprises a predictive output. In some embodiments, diagnosing, predicting, or monitoring the status or outcome is based on the biomedical output comprising one or more molecular classifications and one or more training set assessments. In some embodiments, diagnosing, predicting, or monitoring the status or outcome is based on comparing the similarity of the one or more molecular classifications and the one or more training set assessments. In some embodiments, diagnosing, predicting, or monitoring the status or outcome is definitive when the one or more molecular classifications are similar to the one or more training set assessments. In some embodiments, diagnosing, predicting, or monitoring the status or outcome is indefinite when the one or more molecular classifications contradict the one or more training set assessments. In some embodiments, diagnosing, predicting, or monitoring the status or outcome is indefinite when the one or more molecular classifications are not significant. In some embodiments, diagnosing, predicting, or monitoring further comprises generating one or more biomedical reports. In some embodiments, the one or more biomedical reports comprise information pertaining to the diagnosis, prediction, or monitoring of the status or outcome of the cancer in the subject. In some embodiments, the system further comprises one or more additional software modules configured to transmit the case-specific output, biomedical output, biomedical report, dynamic classifier or a combination thereof. In some embodiments, the case-specific output, biomedical output, biomedical report and/or dynamic classifier are transmitted via a web application. In some embodiments, the web application is implemented as software-as-a-service. In some embodiments, the system further comprises one or more additional software modules configured to add comparator data. In some embodiments, the comparator data comprises a static predictor. In some embodiments, the static predictor is user-selectable. In some embodiments, the static predictor is selected from the group comprising a 21-gene recurrence score, 70-gene Mammaprint signature classifier, and 97-gene genomic grade index (GGI). In some embodiments, the system further comprises one or more additional software modules configured to compare the biomedical output to one or more static outputs, wherein the static outputs are based on one or more static predictors. In some embodiments, the system further comprises one or more additional software modules configured to compare the dynamic classifier to one or more static outputs, wherein the static outputs are based on one or more static predictors.

Also disclosed herein, in some embodiments, are non-transitory computer-readable storage media for use in generating a dynamic classifier. In some embodiments, the non-transitory computer-readable storage media is encoded with a computer program. In some embodiments, the computer program includes instructions executable by a processor to create an application for generating a dynamic classifier. In some embodiments, the storage media comprises (a) a database, in a computer memory, of a plurality of breast cancer cases; (b) a software module configured to receive data input, the data pertaining to a plurality of breast cancer cases; and (c) a software module configured to generate a dynamic classifier, wherein the dynamic classifier comprises a subset of the plurality of breast cancer cases, a subset of the data pertaining to the plurality of breast cancer cases, or a combination thereof. In some embodiments, the storage media comprises one or more additional software modules configured to generate a biomedical output, the biomedical output comprising a comparison of the data of the dynamic classifier to the data of the subject suffering from the breast cancer. In some embodiments, the data input comprises one or more of: case identifiers, gene expression data, clinical survival information, survival annotation, treatment annotation, clinical information, stage of the breast cancer, ethnicity, age, age at diagnosis, age at death, gender, therapeutic regimen, response to a therapeutic regimen, efficacy of a therapeutic regimen, biopsy, clinical tumor staging, tumor pathological staging, lymph node status, or a combination thereof. In some embodiments, the data input comprises gene expression data. In some embodiments, the gene expression data comprises raw gene expression data. In some embodiments, the data input is provided by upload of an output from one or more databases or data sources comprising breast cancer information. In some embodiments, the one or more databases or data sources are selected from a medical records, clinical notes, genomic databases, biomedical databases, clinical trial databases, scientific databases, disease databases, oncogenic databases, biomarker databases, transcriptome databases, mutation databases, epigenomic databases, microbiome databases or a combination thereof. In some embodiments, the one or more databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof. In some embodiments, the data input is provided by manual data entry. In some embodiments, the output from the one or more databases is in one or more formats selected from: a database, a spreadsheet, comma-separated values, tab-separated values, or a combination thereof. In some embodiments, the storage media further comprises one or more additional software modules configured to rank two or more breast cancer cases of the plurality of breast cancer cases. In some embodiments, ranking comprises comparing data of the two or more breast cancer cases to data of the subject. In some embodiments, comparing the data of the two or more breast cancer cases to the data of the subject comprises comparing an expression profile of one or more genes of the two or more breast cancer cases to an expression profile of one or more genes of the subject. In some embodiments, comparing comprises determining the similarity of the two or more breast cancer cases to the subject. In some embodiments, determining the similarity of the two or more breast cancer cases to the subject comprises producing a global similarity matrix over a plurality of genes of the two or more breast cancer cases to a plurality of genes of the subject. In some embodiments, producing the global similarity matrix comprises computing Euclidean distance. In some embodiments, ranking comprises determining molecular similarity of the data of the two or more ranked breast cancer cases to the data of the subject. In some embodiments, the storage media further comprises one or more additional software modules configured to generate a case-specific training subset based on the ranking of the two or more breast cancer cases. In some embodiments, the case-specific training subset comprises a subset of the plurality of breast cancer cases. In some embodiments, the subset of the plurality of breast cancer cases comprises the most similar breast cancer cases to the subject. In some embodiments, the subset of the plurality of breast cancer comprises at least two of the highest ranked breast cancer cases of the two or more ranked breast cancer cases. In some embodiments, the case-specific output comprises the case-specific training subset. In some embodiments, the storage media further comprises one or more additional software modules configured to rank two or more genes of one or more breast cancer cases of the case-specific training subset. In some embodiments, ranking comprises comparing an expression level of the two or more genes of the one or more breast cancer cases to an expression level of two or more genes of the subject. In some embodiments, ranking comprises performing a Kaplan-Meier survival analysis for two or more genes of the one or more breast cancer cases of the case-specific training subset. In some embodiments, ranking is based on one or more of: p-value, hazard ratio, or a combination thereof. In some embodiments, the storage media further comprises one or more additional software modules configured to generate a case-specific gene set based on the ranking of the two or more genes. In some embodiments, the case-specific gene set comprises the subset of the data pertaining to the plurality of breast cancer cases. In some embodiments, the subset of the data comprises one or more of the highest ranked genes. In some embodiments, the case-specific output comprises the case-specific gene set. In some embodiments, the case-specific output is in one or more formats selected from: a database, a spreadsheet, comma-separated values, tab-separated values, or a combination thereof. In some embodiments, the biomedical output comprises one or more molecular classifications. In some embodiments, the one or more molecular classifications are based on a comparison of an average expression level of the one or more highest ranked genes of the case-specific output to an average expression level of one or more genes of the subject. In some embodiments, the biomedical output further comprises one or more training set assessments. In some embodiments, the one or more training set assessments are based on a comparison of the case-specific output to one or more additional subjects suffering from a breast cancer. In some embodiments, the comparison of the case specific output to the one or more additional subjects is based on Kaplan-Meier analysis. In some embodiments, the storage media further comprises one or more additional software modules configured to diagnose, predict, or monitor a status or outcome of the breast cancer in the subject. In some embodiments, diagnosing, predicting or monitoring the status or outcome comprises a prognostic output. In some embodiments, the prognostic output comprises a likelihood of recurrence of the breast cancer in the subject. In some embodiments, the prognostic output comprises a likelihood of lymph node invasion. In some embodiments, the likelihood of lymph node invasion is at the time of diagnosis. In some embodiments, the prognostic output comprises a likelihood of metastasis of the breast cancer in the subject. In some embodiments, diagnosing, predicting or monitoring the status or outcome comprises a predictive output. In some embodiments, diagnosing, predicting, or monitoring the status or outcome is based on the biomedical output comprising one or more molecular classifications and one or more training set assessments. In some embodiments, diagnosing, predicting, or monitoring the status or outcome is based on comparing the similarity of the one or more molecular classifications and the one or more training set assessments. In some embodiments, diagnosing, predicting, or monitoring the status or outcome is definitive when the one or more molecular classifications are similar to the one or more training set assessments. In some embodiments, diagnosing, predicting, or monitoring the status or outcome is indefinite when the one or more molecular classifications contradict the one or more training set assessments. In some embodiments, diagnosing, predicting, or monitoring the status or outcome is indefinite when the one or more molecular classifications are not significant. In some embodiments, diagnosing, predicting, or monitoring further comprises generating one or more biomedical reports. In some embodiments, the one or more biomedical reports comprise information pertaining to the diagnosis, prediction, or monitoring of the status or outcome of the cancer in the subject. In some embodiments, the storage media further comprises one or more additional software modules configured to transmit the case-specific output, biomedical output, biomedical report, dynamic classifier or a combination thereof. In some embodiments, the case-specific output, biomedical output, biomedical report and/or dynamic classifier are transmitted via a web application. In some embodiments, the web application is implemented as software-as-a-service. In some embodiments, the storage media further comprises one or more additional software modules configured to add comparator data. In some embodiments, the comparator data comprises a static predictor. In some embodiments, the static predictor is user-selectable. In some embodiments, the static predictor is selected from the group comprising a 21-gene recurrence score, 70-gene Mammaprint signature classifier, and 97-gene genomic grade index (GGI). In some embodiments, the storage media further comprises one or more additional software modules configured to compare the biomedical output to one or more static outputs, wherein the static outputs are based on one or more static predictors. In some embodiments, the storage media further comprises one or more additional software modules configured to compare the dynamic classifier to one or more static outputs, wherein the static outputs are based on one or more static predictors.

Also disclosed herein are non-transitory computer-readable storage media for use in generating one or more biomedical outputs. In some embodiments, the storage media encoded with a computer program including instructions executable by a processor to create an application comprises (a) a database, in a computer memory, of a plurality of breast cancer cases; (b) a software module configured to receive data input, the data pertaining to a plurality of breast cancer cases; (c) a software module configured to generate a case-specific output, wherein the case specific output comprises a subset of the plurality of breast cancer cases, a subset of the data pertaining to the plurality of breast cancer cases, or a combination thereof; and (d) a software module configured to generate a biomedical output, the biomedical output comprising a comparison of the data of the case-specific output to the data of the subject suffering from the breast cancer. In some embodiments, the data input comprises one or more of: case identifiers, gene expression data, clinical survival information, survival annotation, treatment annotation, clinical information, stage of the breast cancer, ethnicity, age, age at diagnosis, age at death, gender, therapeutic regimen, response to a therapeutic regimen, efficacy of a therapeutic regimen, biopsy, clinical tumor staging, tumor pathological staging, lymph node status, or a combination thereof. In some embodiments, the data input comprises gene expression data. In some embodiments, the gene expression data comprises raw gene expression data. In some embodiments, the data input is provided by upload of an output from one or more databases or data sources comprising breast cancer information. In some embodiments, the one or more databases or data sources are selected from medical records, clinical notes, genomic databases, biomedical databases, clinical trial databases, scientific databases, disease databases, oncogenic databases, biomarker databases, transcriptome databases, mutation databases, epigenomic databases, microbiome databases, or a combination thereof. In some embodiments, the one or more databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof. In some embodiments, the data input is provided by manual data entry. In some embodiments, the output from the one or more databases is in one or more formats selected from: a database, a spreadsheet, comma-separated values, tab-separated values, or a combination thereof. In some embodiments, the storage media further comprises one or more additional software modules configured to rank two or more breast cancer cases of the plurality of breast cancer cases. In some embodiments, ranking comprises comparing data of the two or more breast cancer cases to data of the subject. In some embodiments, comparing the data of the two or more breast cancer cases to the data of the subject comprises comparing an expression profile of one or more genes of the two or more breast cancer cases to an expression profile of one or more genes of the subject. In some embodiments, comparing comprises determining the similarity of the two or more breast cancer cases to the subject. In some embodiments, determining the similarity of the two or more breast cancer cases to the subject comprises producing a global similarity matrix over a plurality of genes of the two or more breast cancer cases to a plurality of genes of the subject. In some embodiments, producing the global similarity matrix comprises computing Euclidean distance. In some embodiments, ranking comprises determining molecular similarity of the data of the two or more ranked breast cancer cases to the data of the subject. In some embodiments, the storage media further comprises one or more additional software modules configured to generate a case-specific training subset based on the ranking of the two or more breast cancer cases. In some embodiments, the case-specific training subset comprises a subset of the plurality of breast cancer cases. In some embodiments, the subset of the plurality of breast cancer cases comprises the most similar breast cancer cases to the subject. In some embodiments, the subset of the plurality of breast cancer comprises at least two of the highest ranked breast cancer cases of the two or more ranked breast cancer cases. In some embodiments, the case-specific output comprises the case-specific training subset. In some embodiments, the storage media further comprises one or more additional software modules configured to rank two or more genes of one or more breast cancer cases of the case-specific training subset. In some embodiments, ranking comprises comparing an expression level of the two or more genes of the one or more breast cancer cases to an expression level of two or more genes of the subject. In some embodiments, ranking comprises performing a Kaplan-Meier survival analysis for two or more genes of the one or more breast cancer cases of the case-specific training subset. In some embodiments, ranking is based on one or more of: p-value, hazard ratio, or a combination thereof. In some embodiments, the storage media further comprises one or more additional software modules configured to generate a case-specific gene set based on the ranking of the two or more genes. In some embodiments, the case-specific gene set comprises the subset of the data pertaining to the plurality of breast cancer cases. In some embodiments, the subset of the data comprises one or more of the highest ranked genes. In some embodiments, the case-specific output comprises the case-specific gene set. In some embodiments, the case-specific output is in one or more formats selected from: a database, a spreadsheet, comma-separated values, tab-separated values, or a combination thereof. In some embodiments, the biomedical output comprises one or more molecular classifications. In some embodiments, the one or more molecular classifications are based on a comparison of an average expression level of the one or more highest ranked genes of the case-specific output to an average expression level of one or more genes of the subject. In some embodiments, the biomedical output further comprises one or more training set assessments. In some embodiments, the one or more training set assessments are based on a comparison of the case-specific output to one or more additional subjects suffering from a breast cancer. In some embodiments, the comparison of the case specific output to the one or more additional subjects is based on Kaplan-Meier analysis. In some embodiments, the storage media further comprises one or more additional software modules configured to diagnose, predict, or monitor a status or outcome of the breast cancer in the subject. In some embodiments, diagnosing, predicting or monitoring the status or outcome comprises a prognostic output. In some embodiments, the prognostic output comprises a likelihood of recurrence of the breast cancer in the subject. In some embodiments, the prognostic output comprises a likelihood of lymph node invasion. In some embodiments, the likelihood of lymph node invasion is at the time of diagnosis. In some embodiments, the prognostic output comprises a likelihood of metastasis of the breast cancer in the subject. In some embodiments, diagnosing, predicting or monitoring the status or outcome comprises a predictive output. In some embodiments, diagnosing, predicting, or monitoring the status or outcome is based on the biomedical output comprising one or more molecular classifications and one or more training set assessments. In some embodiments, diagnosing, predicting, or monitoring the status or outcome is based on comparing the similarity of the one or more molecular classifications and the one or more training set assessments. In some embodiments, diagnosing, predicting, or monitoring the status or outcome is definitive when the one or more molecular classifications are similar to the one or more training set assessments. In some embodiments, diagnosing, predicting, or monitoring the status or outcome is indefinite when the one or more molecular classifications contradict the one or more training set assessments. In some embodiments, diagnosing, predicting, or monitoring the status or outcome is indefinite when the one or more molecular classifications are not significant. In some embodiments, diagnosing, predicting, or monitoring further comprises generating one or more biomedical reports. In some embodiments, the one or more biomedical reports comprise information pertaining to the diagnosis, prediction, or monitoring of the status or outcome of the cancer in the subject. In some embodiments, the storage media further comprises one or more additional software modules configured to transmit the case-specific output, biomedical output, biomedical report, dynamic classifier or a combination thereof. In some embodiments, the case-specific output, biomedical output, biomedical report and/or dynamic classifier are transmitted via a web application. In some embodiments, the web application is implemented as software-as-a-service. In some embodiments, the storage media further comprises one or more additional software modules configured to add comparator data. In some embodiments, the comparator data comprises a static predictor. In some embodiments, the static predictor is user-selectable. In some embodiments, the static predictor is selected from the group comprising a 21-gene recurrence score, 70-gene Mammaprint signature classifier, and 97-gene genomic grade index (GGI). In some embodiments, the storage media further comprises one or more additional software modules configured to compare the biomedical output to one or more static outputs, wherein the static outputs are based on one or more static predictors. In some embodiments, the storage media further comprises one or more additional software modules configured to compare the dynamic classifier to one or more static outputs, wherein the static outputs are based on one or more static predictors.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary workflow for a dynamic predictor/prognosticator method.

FIG. 2A-D shows survival curves for the dynamic classifier and genomic surrogates of three commercially available prognostic signatures applied to the same 3,534 cases. The dynamic re-training was computed using the top 25 genes and a training set size of 400 samples. (A) 21-gene score; (B) Genomic grade index; (C) 70-gene signature; and (D) Dynamic re-training.

FIG. 3A-D shows survival curves for the dynamic classifier and genomic surrogates of three commercially available prognostic signatures applied to the ER positive and HER2 negative patients (untreated). (A) 21-gene score; (B) Genomic grade index; (C) 70-gene signature; and (D) Dynamic re-training.

FIG. 4A-D shows survival curves for the dynamic classifier and genomic surrogates of three commercially available prognostic signatures applied to the ER positive and HER2 negative patients (treated). (A) 21-gene score; (B) Genomic grade index; (C) 70-gene signature; and (D) Dynamic re-training.

FIG. 5A-C shows survival curves for the dynamic classifier and genomic surrogates of three commercially available prognostic signatures applied to the ER negative and HER2 negative patients (treated). (A) 21-gene score; (B) Genomic grade index; and (C) Dynamic re-training.

FIG. 6A-D shows survival curves for the dynamic classifier and genomic surrogates of three commercially available prognostic signatures applied to the HER2 positive patients. (A) 21-gene score; (B) Genomic grade index; (C) 70-gene signature; and (D) Dynamic re-training.

FIG. 7A-E shows performance of the dynamic classifier and three other prognostic signatures in 325 independent validation samples that were not included in the pool of 3,534 samples used for selection of the training set samples. (A) Dynamic re-training (all patients); (B) Dynamic retraining—chemotherapy patients only; (C) 70-gene signature; and (D) 21-gene score; (E) Genomic grade index.

### DETAILED DESCRIPTION OF THE INVENTION

Described herein, in certain embodiments, are computer-implemented methods for generating dynamic classifiers. In some embodiments, the dynamic classifiers are case-specific. Additionally, in some instances, the dynamic classifiers are based on comparative analysis of a plurality of cancer cases to a cancer in a subject. In some embodiments, the method for generating a dynamic classifier comprises (a) receiving, by a computer, data input, the data pertaining to a plurality of cancer cases; and (b) generating, by the computer, a dynamic classifier, wherein the dynamic classifier is based on a comparison of the data pertaining to the plurality of cancer cases to data pertaining to a subject suffering from a cancer. In some embodiments, the dynamic classifier comprises a subset of the plurality of cancer cases. Alternatively, or additionally, the dynamic classifier comprises a subset of the data pertaining to the plurality of cancer cases. In some embodiments, the dynamic classifiers are used to provide a prognostic output. In other instances, the dynamic classifiers are used to provide a predictive output. In some embodiments, the cancer is a breast cancer.

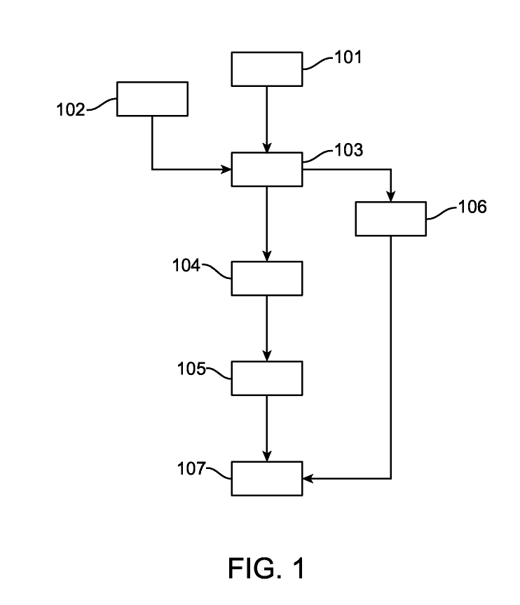

Also described herein, in certain embodiments, are computer-implemented methods for diagnosing, predicting, or monitoring a status or outcome of a cancer in a subject in need thereof. Generally, the computer-implemented methods comprise (a) receiving, by a computer, data input, the data pertaining to a plurality of cancer cases; (b) generating, by the computer, a case-specific output, wherein the case-specific output comprises a subset of the plurality of cancer cases, a subset of the data pertaining to the plurality of cancer cases, or a combination thereof, and wherein the case-specific output is based on a comparison of the data pertaining to the plurality of cancer cases to data pertaining to a subject suffering from a cancer; and (c) generating, by the computer, a biomedical output, the biomedical output comprising a comparison of the data of the case-specific output to the data of the subject suffering from the cancer. In some embodiments, the method further comprises diagnosing, predicting or monitoring, by the computer, a status or outcome of the cancer in the subject based on the biomedical output. In some embodiments, the cancer is a breast cancer. An exemplary workflow is depicted in FIG. 1. As shown in FIG. 1, a large database (101) is used to select a subset of training cases (e.g., case-specific output or case-specific training subset) (103) that are molecularly the most similar to the test cases (e.g., subject-case or subject suffering from a cancer) (102). In some embodiments, the training subset (103) is used to identify predictive features (e.g., genes or case-specific gene set) (104) and to develop the test-case specific predictor (e.g., dynamic classifier or biomedical output) (107). In some embodiments, the method further comprises assessing the training set (106). In some embodiments, assessing the training set comprises comparison of the training set to a plurality of cancer cases (e.g., a plurality of subjects suffering from a cancer, a plurality of the cancer cases). In some embodiments, the method comprises molecular classification (105). In some embodiments, molecular classification comprises a comparison of data from the subject suffering from a cancer to the data from the training subset.

Also disclosed herein, in some embodiments, are dynamic computer-implemented systems for generating dynamic classifiers. In some instances, the system comprises (a) a digital processing device comprising an operating system configured to perform executable instructions and a memory device; and (b) a computer program including instructions executable by the digital processing device to create an application comprising: (i) a software module configured to receive data input, the data pertaining to a plurality of cancer cases; and (ii) a software module configured to generate a dynamic classifier. In some embodiments, the dynamic classifier comprises a subset of the plurality of cancer cases, a subset of the data pertaining to the plurality of cancer cases, or a combination thereof. In some embodiments, generating the dynamic classifier comprises comparing the data pertaining to the plurality of cancer cases to the data pertaining to a subject suffering from a cancer. In some embodiments, the system further comprises one or more additional software modules configured to generate a biomedical output. In some embodiments, the biomedical output comprises a comparison of the data of the dynamic classifier to the data of the subject suffering from the cancer. In some embodiments, the cancer is a breast cancer.

Further disclosed herein, in some embodiments, are dynamic computer-implemented systems for diagnosing, predicting, or monitoring a status or outcome of a cancer in a subject in thereof. In some instances, the system comprises (a) a digital processing device comprising an operating system configured to perform executable instructions and a memory device; and (b) a computer program including instructions executable by the digital processing device to create an application comprising: (i) a software module configured to receive data input, the data pertaining to a plurality of cancer cases; (ii) a software module configured to generate a case-specific output, wherein the case specific output comprises a subset of the plurality of cancer cases, a subset of the data pertaining to the plurality of cancer cases, or a combination thereof; and (iii) a software module configured to generate a biomedical output, the biomedical output comprising a comparison of the data of the case-specific output to the data of the subject suffering from the cancer. In some embodiments, the cancer is a breast cancer.

Also disclosed herein, in some embodiments, are non-transitory computer-readable storage media for use in generating a dynamic classifier. In some embodiments, the non-transitory computer-readable storage media is encoded with a computer program. In some embodiments, the computer program includes instructions executable by a processor to create an application for generating a dynamic classifier. In some embodiments, the storage media comprises (a) a database, in a computer memory, of a plurality of cancer cases; (b) a software module configured to receive data input, the data pertaining to a plurality of cancer cases; and (c) a software module configured to generate a dynamic classifier, wherein the dynamic classifier comprises a subset of the plurality of cancer cases, a subset of the data pertaining to the plurality of cancer cases, or a combination thereof. In some embodiments, the storage media comprises one or more additional software modules configured to generate a biomedical output, the biomedical output comprising a comparison of the data of the dynamic classifier to the data of the subject suffering from the cancer. In some embodiments, the cancer is a breast cancer.

Also disclosed herein, in some embodiments, are non-transitory computer-readable storage media for use in diagnosing, predicting or monitoring a status or outcome of a cancer in a subject in need thereof. In some embodiments, the non-transitory computer-readable storage media is encoded with a computer program. In some embodiments, the computer program includes instructions executable by a processor to create an application for diagnosing, predicting or monitoring a status or outcome of a cancer in a subject in need thereof. In some embodiments, the application comprises (a) a database, in a computer memory, of a plurality of cancer cases; (b) a software module configured to receive data input, the data pertaining to a plurality of cancer cases; (c) a software module configured to generate a case-specific output, wherein the case specific output comprises a subset of the plurality of cancer cases, a subset of the data pertaining to the plurality of cancer cases, or a combination thereof; and (d) a software module configured to generate a biomedical output, the biomedical output comprising a comparison of the data of the case-specific output to the data of the subject suffering from the cancer. In some embodiments, the cancer is a breast cancer.

### CERTAIN DEFINITIONS

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

### Cancer Data

In some embodiments, the systems, media, and methods described herein utilize cancer data. As used herein, the term “cancer data” refers to data pertaining to one or more cancers. In further embodiments, the cancer data is suitably aggregate data. In other embodiments, the cancer data is suitably individual data. In further embodiments, the cancer data pertains to individuals. In still further embodiments, the cancer data pertains to a plurality of cancer cases. The cancer data suitably pertains to individuals of various ancestral backgrounds. By way of non-limiting examples, the cancer data suitably pertains to individuals of Caucasian, African, Asian, Latino, Native American descent, and the like. In some embodiments, the cancer data pertains to individuals of European, Eastern European, French, German, Italian, Spanish, Portuguese, Russian, Romanian, African American, African, Mexican, Puerto Rican, Dominican, Filipino, Chinese, Japanese, Vietnamese, Taiwanese descent, and the like. In some embodiments, the cancer data pertains to individuals of various ages. For example, the data pertains to individuals less than about 90, 80, 70, 60, 50, 40, 30, 20, 10 years old, or a combination thereof. In another example, the data pertains to individuals at least about 5, 10, 15, 20, 25, 30, 35, 40, 45, 50, 55, 60, 65, 70, 75, 80, 85, 90 years old, or a combination thereof. In some embodiments, the cancer data pertains to individuals with various stages of cancer. In some embodiments, the cancer data pertains to individuals with Stage 0, Stage I, Stage II, Stage IIIA, Stage IIIB, Stage IIIC, Stage IV cancer, or a combination thereof.

Many types of cancer data are suitable. In some embodiments, the data input comprises one or more of: case identifiers, gene expression data, clinical survival information, survival annotation, treatment annotation, clinical information, stage of the cancer, ethnicity, age, age at diagnosis, age at death, gender, therapeutic regimen, response to a therapeutic regimen, efficacy of a therapeutic regimen, biopsy, clinical tumor staging, tumor pathological staging, lymph node status, or a combination thereof. In some embodiments, suitable cancer data comprises case identifiers. In further embodiments, case identifiers comprise numeric and alphanumeric identifiers used by, for example, analysts, medical personnel or software to refer to individuals, data sets, databases, source, or a combination thereof.

In some embodiments, the cancer data comprises gene expression data. In some embodiments, the gene expression data comprises raw gene expression data. In some embodiments, the gene expression data is generated on a HG-U133A (GPL2) array, HG-U133 Plus 2.0 (GPL570) array, or a combination thereof. In some embodiments, the cancer data comprises gene expression data from one or more data sets. In some embodiments, the one or more data sets comprise gene expression data from at least 30 individual cases. In some embodiments, the cancer data comprises gene expression data from at least about 50, 100, 200, 300, 400, 500, 600, 700, 800, 900, 1000 or more individual cases from one or more data sets. In some embodiments, the cancer data comprises gene expression data from at least about 100 individual cases. In some embodiments, the cancer data comprises gene expression data from at least about 200 individual cases. In some embodiments, the cancer data comprises gene expression data from at least about 300 individual cases. In some embodiments, the cancer data comprises gene expression data from at least about 400 individual cases. In some embodiments, the cancer data comprises gene expression data from at least about 500 individual cases. In some embodiments, the cancer data comprises gene expression data from at least about 1500, 2000, 2500, 3000, 3500, 4000, 4500, 5000, 5500, 6000 or more individual cases from one or more data sets. In some embodiments, the cancer data comprises gene expression data from at least about 5, 10, 15, 20, 25 or more data sets. In some embodiments, the cancer data comprises gene expression data from at least about 5 or more data sets. In some embodiments, the cancer data comprises gene expression data from at least about 10 or more data sets. In some embodiments, the cancer data comprises gene expression data for at least about 1, 3, 5, 10, 15, 20, 25, 30, 35, 40, 45, 50, 55, 60, 65, 70, 75, 80, 85, 90, 95, 100 or more genes. In some embodiments, the cancer data comprises gene expression data for at least about 125, 150, 175, 200, 225, 250, 275, 300, 325, 350, 375, 400, 450, 500 or more genes. In some embodiments, the cancer data comprises gene expression data for at least about 600, 700, 800, 900, 1000, 2000, 3000, 4000, 5000, 6000, 7000, 8000, 9000 or more genes. In some embodiments, the cancer data comprises gene expression data for at least about 10,000; 12,500; 15,000; 17,500; 20,000; 22,500; 25,000 or more genes. In some embodiments, the cancer data comprises gene expression data for at least about 3 or more genes. In some embodiments, the cancer data comprises gene expression data for at least about 5 or more genes. In some embodiments, the cancer data comprises gene expression data for at least about 10 or more genes. In some embodiments, the cancer data comprises gene expression data for at least about 15 or more genes. In some embodiments, the cancer data comprises gene expression data for at least about 20 or more genes. In some embodiments, the cancer data comprises gene expression data for at least about 25 or more genes. In some embodiments, the cancer data comprises gene expression data for at least about 30 or more genes. In some embodiments, the cancer data comprises gene expression data for at least about 50 or more genes.

In some embodiments, the cancer data comprises medical or health-related information. In some embodiments, medical or health-related information comprises medical history. In some embodiments, medical or health-related information comprises pre-existing medical conditions, therapeutic regimens, response to a therapeutic regimen, efficacy of a therapeutic regimen, dosage information, surgery, biopsy, survival information, clinical survival information, relapse-free survival information, survival annotation, treatment annotation, clinical information, relapse information, stage of the cancer, disease progression, age at diagnosis, age at death, age at relapse, or a combination thereof.

In some embodiments, suitable cancer data comprises demographic information. In further embodiments, demographic information comprises ethnicity, education, age, gender, location, marital status, children, employment, income, and the like.

In some embodiments, the systems, media, and methods described herein include a software module configured to receive input of cancer data. In further embodiments, the data input is provided by manual data entry. In various embodiments, manual data entry is achieved, for example, by typing, pointing device, touchscreen, voice recognition, and the like. In other embodiments, the data input is provided by upload of an output from one or more cancer information applications. In other embodiments, the data input is provided by upload of an output from one or more databases. In some embodiments, the one or more databases comprise genome, transcriptome, pharmacogenomic, pharmacodynamic databases, or a combination thereof. In further embodiments, the data input is provided by upload of an output from databases or data sources by, for example, medical records, clinical notes, genomic databases, biomedical databases, clinical trial databases, scientific databases, disease databases, oncogenic databases, biomarker databases, transcriptome databases, mutation databases, epigenomic databases, microbiome databases, or a combination thereof. In some embodiments, the databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof. In some embodiments, the data input is provided by manual data entry

In some instances, the data input is provided in any suitable format. In still further embodiments, the data input is provided in a format such as a database, a spreadsheet, comma-separated values (CSV), and tab-separated values (TSV), Extensible Markup Language (XML), and the like.

### Case Tagging

In some embodiments, the systems, media, and methods described herein utilize data tagging. As used herein in some embodiments, “tagging” refers to associating a piece of information with metadata to facilitate efficient organization, filtering, browsing, or searching. In further embodiments, the tagging is molecular tagging and the metadata associates the information with a molecular similarity to cancer case of a subject. In still further embodiments, molecular tagging facilitates analysis, filtering, searching, identification, and quantification of discrepancies, disparities, and inequalities in cancer data based on molecular or gene expression profiles.

Molecular tagging is suitably achieved in a variety of ways. In some embodiments, molecular tagging is achieved manually. In further embodiments, a human analyst associates cancer data with the cancer case to which it pertains. In various embodiments, a human analyst utilizes cues for gene expression data or gene expression profile to tag data based on molecular similarity to the subject-specific cancer case.

In other embodiments, software associates cancer data with the cancer case to which it pertains. In further embodiments, the systems, media, and methods described herein include a software module configured to tag cancer data with a molecular match to cancer data pertaining to a subject. In various embodiments, a software module utilizes cross-references to gene expression data, survival annotation, treatment annotation, stage of the cancer, and the like to tag data based on molecular similarity to a subject-specific cancer case.

### Data Ranking

In some embodiments, the systems, media, and methods described herein utilize data ranking. As used herein in some embodiments, “ranking” refers to sorting a piece of information with metadata to facilitate efficient organization, filtering, browsing, or searching.

In some embodiments, the systems, media and methods further comprise ranking two or more cancer cases of a plurality of cancer cases. In some embodiments, ranking comprises comparing data of the two or more cancer cases to data of the subject. In some embodiments, comparing the data of the two or more cancer cases to the data of the subject comprises comparing an expression profile of one or more genes of the two or more cancer cases to an expression profile of one or more genes of the subject. In some embodiments, comparing further comprises determining the similarity of the two or more cancer cases to the subject. In some embodiments, determining the similarity of the two or more cancer cases to the subject comprises producing a global similarity matrix over a plurality of genes of the two or more cancer cases to a plurality of genes of the subject. In some embodiments, producing the global similarity matrix comprises computing Euclidean distance. In some embodiments, ranking comprises determining molecular similarity of the data of the two or more ranked cancer cases to the data of the subject.

In some embodiments, the systems, media and methods disclosed herein further comprise producing a case-specific training subset based on the ranking of the two or more cancer cases. In some embodiments, producing the case-specific training subset comprises selecting a subset of the highest ranked cancer cases. In some embodiments, the case-specific training subset comprises a subset of the plurality of cancer cases. In some embodiments, the subset of the plurality of cancer cases comprises the most similar cancer cases to the subject. In some embodiments, the subset of the plurality of cancer comprises at least two of the highest ranked cancer cases of the two or more ranked cancer cases. In some embodiments, the case-specific training subset comprises at least about 2, 5, 10, 15, 20, 25, 30, 35, 40, 45, 50, 60, 70, 80, 90, or 100 of the highest ranked cancer cases. In some embodiments, the case-specific training subset comprises at least about 125, 150, 175, 200, 225, 250, 275, 300, 325, 350, 375, 400, 425, 450, 475, 500, 550, 600, 650, 700, 750, 800, 850, 900, 950 or 1000 of the highest ranked cancer cases. In some embodiments, the case-specific training subset comprises at least about 100 of the highest ranked cancer cases. In some embodiments, the case-specific training subset comprises at least about 200 of the highest ranked cancer cases. In some embodiments, the case-specific training subset comprises at least about 300 of the highest ranked cancer cases. In some embodiments, the case-specific training subset comprises at least about 400 of the highest ranked cancer cases. In some embodiments, the case-specific training subset comprises less than about 1000, 900, 800, 700, 650, 600, 550, 500, 450, 400, 350, 300, 250, 200, or 100 of the highest ranked cancer cases. In some embodiments, the case-specific training subset comprises less than about 800 of the highest ranked cancer cases. In some embodiments, the case-specific training subset comprises less than about 600 of the highest ranked cancer cases. In some embodiments, the case-specific training subset comprises less than about 500 of the highest ranked cancer cases. In some embodiments, the case-specific training subset comprises between about 50 to about 1000 of the highest ranked cancer cases. In some embodiments, the case-specific training subset comprises between about 50 to about 750 of the highest ranked cancer cases. In some embodiments, the case-specific training subset comprises between about 50 to about 600 of the highest ranked cancer cases. In some embodiments, the case-specific training subset comprises between about 100 to about 1000 of the highest ranked cancer cases. In some embodiments, the case-specific training subset comprises between about 100 to about 750 of the highest ranked cancer cases. In some embodiments, the case-specific training subset comprises between about 100 to about 600 of the highest ranked cancer cases. In some embodiments, the case-specific output comprises the case-specific training subset. In some embodiments, the case-specific training subset is in one or more formats selected from: a database, a spreadsheet, comma-separated values, tab-separated values, or a combination thereof. In some embodiments, the case-specific training subset is in the form of a database. In some embodiments, the case-specific training subset is in the form of a spreadsheet.

In some embodiments, the systems, media and methods disclosed herein further comprise ranking two or more genes of one or more cancer cases of the case-specific training subset. In some embodiments, ranking comprises comparing an expression level of the two or more genes of the one or more cancer cases to an expression level of two or more genes of the subject. In some embodiments, ranking comprises performing a Kaplan-Meier survival analysis for two or more genes of the one or more cancer cases of the case-specific training subset. In some embodiments, ranking is based on one or more of: p-value, hazard ratio, or a combination thereof. In some embodiments, ranking comprises tagging one or more cancer cases with a similarity to a cancer in a subject.

In some embodiments, the systems, media and methods disclosed herein further comprise producing a case-specific gene set based on the ranking of the two or more genes. In some embodiments, producing the case-specific gene set comprises selected a subset of the highest ranked genes. In some embodiments, the case-specific gene set comprises the subset of the data pertaining to the plurality of cancer cases. In some embodiments, the subset of the data comprises one or more of the highest ranked genes. In some embodiments, the case-specific gene set comprises at least about 2, 5, 10, 15, 20, 25, 30, 35, 40, 45, 50, 60, 70, 80, 90, or 100 of the highest ranked genes. In some embodiments, the case-specific gene set comprises at least about 125, 150, 175, 200, 225, 250, 275, 300, 325, 350, 375, 400, 425, 450, 475, 500, 550, 600, 650, 700, 750, 800, 850, 900, 950 or 1000 of the highest ranked genes. In some embodiments, the case-specific gene set comprises at least about 5 of the highest ranked genes. In some embodiments, the case-specific gene set comprises at least about 10 of the highest ranked genes. In some embodiments, the case-specific gene set comprises at least about 25 of the highest ranked genes. In some embodiments, the case-specific gene set comprises less than about 500, 450, 400, 350, 300, 250, 200, or 100 of the highest ranked genes. In some embodiments, the case-specific gene set comprises less than about 90, 80, 70, 60, 50, 45, 40, 35, 30, 25, 20, 15, or 10 of the highest ranked genes. In some embodiments, the case-specific gene set comprises less than about 100 of the highest ranked genes. In some embodiments, the case-specific gene set comprises less than about 50 of the highest ranked genes. In some embodiments, the case-specific gene set comprises less than about 40 of the highest ranked genes. In some embodiments, the case-specific gene set comprises between about 5 to about 100 of the highest ranked genes. In some embodiments, the case-specific gene set comprises between about 5 to about 75 of the highest ranked genes. In some embodiments, the case-specific gene set comprises between about 5 to about 50 of the highest ranked genes. In some embodiments, the case-specific gene set comprises between about 10 to about 100 of the highest ranked genes. In some embodiments, the case-specific gene set comprises between about 10 to about 50 of the highest ranked genes. In some embodiments, the case-specific gene set comprises between about 20 to about 50 of the highest ranked genes. In some embodiments, the case-specific output comprises the case-specific gene set. In some embodiments, the case-specific gene set is in one or more formats selected from: a database, a spreadsheet, comma-separated values, tab-separated values, or a combination thereof. In some embodiments, the case-specific gene set is in the form of a database. In some embodiments, the case-specific gene set is in the form of a spreadsheet.

In some embodiments, the highest ranked genes are expressed in at least about 5%, 10%, 15%, 20%, 25%, 30%, 35%, 40%, 45%, 50%, 55%, 60%, 65%, 70%, 75%, 80%, 85%, 90%, 95%, 97% or more of the cancer cases. In some embodiments, the highest ranked genes are expressed in at least about 25% of the cancer cases. In some embodiments, the highest ranked genes are expressed in at least about 30% of the cancer cases. In some embodiments, the highest ranked genes are expressed in at least about 35% of the cancer cases. In some embodiments, the highest ranked genes are expressed in at least about 5%, 10%, 15%, 20%, 25%, 30%, 35%, 40%, 45%, 50%, 55%, 60%, 65%, 70%, 75%, 80%, 85%, 90%, 95%, 97% or more of the cancer cases of the case-specific output. In some embodiments, the highest ranked genes are expressed in at least about 25% of the cancer cases of the case-specific output. In some embodiments, the highest ranked genes are expressed in at least about 30% of the cancer cases of the case-specific output. In some embodiments, the highest ranked genes are expressed in at least about 35% of the cancer cases of the case-specific output. In some embodiments, the highest ranked genes are expressed in at least about 5%, 10%, 15%, 20%, 25%, 30%, 35%, 40%, 45%, 50%, 55%, 60%, 65%, 70%, 75%, 80%, 85%, 90%, 95%, 97% or more of the cancer cases of the case-specific training subset. In some embodiments, the highest ranked genes are expressed in at least about 25% of the cancer cases of the case-specific training subset. In some embodiments, the highest ranked genes are expressed in at least about 30% of the cancer cases of the case-specific training subset. In some embodiments, the highest ranked genes are expressed in at least about 35% of the cancer cases of the case-specific training subset.

### Biomedical Output

In some embodiments, the systems, media and methods disclosed herein comprise one or more biomedical outputs or uses thereof. In some embodiments, the biomedical output comprises one or more molecular classifications. In some embodiments, the one or more molecular classifications are based on a comparison of an average expression level of the one or more highest ranked genes of the case-specific output to an average expression level of one or more genes of the subject. In some embodiments, the biomedical output further comprises one or more training set assessments. In some embodiments, the one or more training set assessments are based on a comparison of the case-specific output to one or more additional subjects suffering from a cancer. In some embodiments, the comparison of the case specific output to the one or more additional subjects is based on Kaplan-Meier analysis.

### Dynamic Classifier

Further disclosed herein in some embodiments are systems, media and methods for generating one or more dynamic classifiers. In some embodiments, the one or more dynamic classifiers are generated by (a) comparing data input from a plurality of cancer cases to data input from a subject suffering from a cancer; (b) selecting a subset of the plurality of cancer cases to produce a case-specific output, wherein selecting is based on the comparison of the data input from the plurality of cancer cases to the data input from the subject; (c) comparing an expression profile of one or more genes from the case-specific output to an expression profile of one or more genes from the data input from the subject; and (d) generating one or more dynamic classifiers comprising one or more genes, wherein generating the one or more dynamic classifiers is based on the comparison of the expression profile from the case-specific output to the expression profile from the data input from the subject.

In some embodiments, the one or more dynamic classifiers comprise a case-specific output, biomedical output, or a combination thereof. In some embodiments, the one or more dynamic classifiers are based on a case-specific output, biomedical output, or a combination thereof. In some embodiments, the one or more dynamic classifiers comprise one or more genes. In some embodiments, the one or more genes are selected from one or more genes from a case-specific output, biomedical output, or a combination thereof. In some embodiments, the one or more dynamic classifiers are based on a comparison of data from a data input, case-specific output, and biomedical output to data from a subject suffering from a cancer. In some embodiments, the dynamic classifier comprises at least about 1, 2, 3, 4, 5, 6, 7, 8, 9, 10, 15, 20, 25, 30, 35, 40, 45, 50, 55, 60, 65, 70, 75, 80, 85, 90, 95, 100 or more genes. In some embodiments, the genes are selected based on molecular similarity of an expression profile of the genes from the data input, case-specific output, and/or biomedical output to an expression profile of the genes from a subject-specific cancer case. In some embodiments, the one or more dynamic classifiers are unique to a specific subject suffering from a cancer.

In some embodiments, the systems, media and methods described herein comprise one or more dynamic classifiers or uses thereof. In some embodiments, the one or more dynamic classifiers are used to diagnose, predict, or monitor a status or outcome of cancer in a subject in need thereof.

### Data Display

In some embodiments, the systems, media, and methods described herein include a data display, or use of the same. In further embodiments, a data display presents cancer data. In still further embodiments, a data display presents a comparison of cancer data based on molecular similarity to a subject-specific cancer case. In still further embodiments, a data display presents a comparison of cancer data based on a gene expression profile. In various embodiments, a comparison of cancer data based on molecular similarity is suitably presented in narrative form (e.g., text descriptions, etc.), numeric form (e.g., scores, rankings, ratings, percentages, etc.), graphic form (e.g., charts, tables, graphs, heat maps, etc.), or combinations thereof.

In some embodiments, a data display is based on a subset of the cancer data available. For example, in various further embodiments, a data display is based on application of a filter to the cancer data available. In some embodiments, a data display is based on a user configurable subset of the cancer data. In further embodiments, a data display presents a subset of the cancer data filtered based on time. For example, in particular embodiments, a data display presents cancer data for one or more particular years, one or more particular quarters, one or more particular months, and the like. In further embodiments, a data display presents a subset of the cancer data filtered based on molecular similarity to a subject-specific cancer case.

In some embodiments, the systems, media, and methods described herein include a software module configured to generate a display of the data the display comprising comparison of the data based on molecular similarity to a subject-specific cancer case, the comparison in numeric and graphic form.

### Comparators

In some embodiments, the systems, media, and methods described herein include comparators, or use of the same. In further embodiments, a data display presents a case-specific output, biomedical output, biomedical report, and/or dynamic classifier and further presents a comparison with a comparator predictor. In some embodiments, the comparator predictor is a static predictor. In some embodiments, the static predictor comprises a 21-gene recurrence score, 70-gene Mammaprint signature classifier, 97-gene genomic grade index (GGI), or a combination thereof. In further embodiments, the static predictor is user-selectable. In other embodiments, the static predictor is selected based on the characteristics of the cancer, subject, or output.

In some embodiments, the systems, media and methods described herein further comprise comparing a biomedical output or dynamic classifier to one or more static outputs, wherein the static outputs are based one or more static predictors. In some embodiments, the static predictor comprises a 21-gene recurrence score, 70-gene Mammaprint signature classifier, 97-gene genomic grade index (GGI), or a combination thereof. In further embodiments, the static predictor is user-selectable.

### Digital Processing Device

In some embodiments, the systems, media, and methods described herein include a digital processing device, or use of the same. In further embodiments, the digital processing device includes one or more hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, the digital processing device includes an input device to receive information from a user. In some embodiments, the user is a subject suffering from a cancer, medical professional, researcher, analyst, or a combination thereof. In some embodiments, the medical professional is a doctor, nurse, physician's assistant, pharmacist, medical consultant, or other hospital or medical personnel. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

### Non-Transitory Computer Readable Storage Medium

In some embodiments, the systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the systems, media, and methods disclosed herein include one or more databases, data sources, or use of the same. In view of the disclosure provided herein, those of skill in the art will recognize that many databases are suitable for storage and retrieval of cancer data. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

In some embodiments, the databases or data sources are selected from medical records, clinical notes, genomic databases, biomedical databases, clinical trial databases, scientific databases, disease databases, oncogenic databases, biomarker databases, transcriptome databases, mutation databases, epigenomic databases, microbiome databases, or a combination thereof. In some embodiments, the one or more databases or sources comprise publicly available databases, proprietary databases, or a combination thereof. In some embodiments, the publicly available databases comprise GEO database, Pubmed, clinicaltrials.gov, Orphanet, Human Phenotype Ontology (HPO), Online Mendelian Inheritance in Man (OMIM), Model Organism Gene Knock-Out databases, Kegg Disease Database, Cancer Genome Project, GeneCards, or a combination thereof.

### Data Transmission

In some embodiments, the systems, media and methods disclosed herein further comprise transmission of the case-specific output, biomedical output, biomedical report, dynamic classifier or a combination thereof. In some embodiments, the outputs, reports, and/or classifiers are transmitted electronically. In some embodiments, the case-specific output, biomedical output, biomedical report and/or dynamic classifiers are transmitted via a web application. In some embodiments, the web application is implemented as software-as-a-service.

In some embodiments, the systems, media and methods disclosed herein further comprise one or more transmission devices comprising an output means for transmitting one or more data, results, outputs, information, biomedical outputs, biomedical reports and/or dynamic classifiers. In some embodiments, the output means takes any form which transmits the data, results, requests, and/or information and comprises a monitor, printed format, printer, computer, processor, memory location, or a combination thereof. In some embodiments, the transmission device comprises one or more processors, computers, and/or computer systems for transmitting information.

In some embodiments, transmission comprises tangible transmission media and/or carrier-wave transmission media. In some embodiments, tangible transmission media include coaxial cables; copper wire and fiber optics, including the wires that comprise a bus within a computer system. In some embodiments, carrier-wave transmission media takes the form of electric or electromagnetic signals, or acoustic or light waves such as those generated during radio frequency (RF) and infrared (IR) data communications.

In some embodiments, the outputs, reports, and/or classifiers are transmitted to one or more users. In some embodiments, the one or more users are a subject suffering from a cancer, medical professional, researcher, analyst, or a combination thereof. In some embodiments, the medical professional is a doctor, nurse, physician's assistant, pharmacist, medical consultant, or other hospital or medical personnel.

### Exemplary Uses and Applications

In some embodiments, the systems, media and methods disclosed herein are used to diagnose, predict or monitor a status or outcome of a cancer in a subject in need thereof. In some embodiments, diagnosing, predicting or monitoring the status or outcome comprises a prognostic output. In some embodiments, the prognostic output comprises a likelihood of recurrence of the cancer in the subject. In some embodiments, the prognostic output comprises a likelihood of lymph node invasion. In some embodiments, the likelihood of lymph node invasion is at the time of diagnosis. In some embodiments, the prognostic output comprises a likelihood of metastasis of the cancer in the subject. In some embodiments, diagnosing, predicting or monitoring the status or outcome comprises a predictive output. In some embodiments, the predictive output comprises predicting a response of the subject to a therapeutic regimen.

In some embodiments, diagnosing, predicting or monitoring the status or outcome comprises determining a stage of the cancer in the subject. In some embodiments, diagnosing, predicting or monitoring the status or outcome comprises treating the cancer in the subject. In some embodiments, diagnosing, predicting or monitoring the status or outcome comprises determining, modifying, or maintaining a therapeutic regimen. In some embodiments, modifying a therapeutic regimen comprises increasing, decreasing, terminating, or otherwise altering a therapeutic regimen. In some embodiments, modifying a therapeutic regimen comprises increasing, decreasing, or adjusting a dosage or frequency of dosage of one or more anti-cancer agents of a therapeutic regimen. In some embodiments, modifying a therapeutic regimen comprises adding one or more anti-cancer agents to a therapeutic regimen. In some embodiments, modifying a therapeutic regimen comprises removing one or more anti-cancer agents from a therapeutic regimen. In some embodiments, diagnosing, predicting or monitoring the status or outcome comprises administering a therapeutic regimen.

In some embodiments, diagnosing, predicting, or monitoring the status or outcome is based on the biomedical output comprising the one or more molecular classifications and one or more training set assessments. In some embodiments, diagnosing, predicting, or monitoring the status or outcome is based on comparing the similarity of the one or more molecular classifications and the one or more training set assessments. In some embodiments, diagnosing, predicting, or monitoring the status or outcome is definitive when the one or more molecular classifications are similar to the one or more training set assessments. In some embodiments, diagnosing, predicting, or monitoring the status or outcome is indefinite when the one or more molecular classifications contradict the one or more training set assessments. In some embodiments, diagnosing, predicting, or monitoring the status or outcome is indefinite when the one or more molecular classifications are not significant. In some embodiments, diagnosing, predicting, or monitoring further comprises generating one or more biomedical reports. In some embodiments, the one or more biomedical reports comprise information pertaining to the diagnosis, prediction, or monitoring of the status or outcome of the cancer in the subject.

In some embodiments, the systems, media and/or methods disclosed herein are used to diagnose, predict or monitor a status or outcome of a cancer in a subject in need thereof. In some embodiments, the systems, media and/or methods have a hazard ratio (HR) of at least about 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.90 or more. In some embodiments, the systems, media and/or methods have a hazard ratio (HR) of greater than about 3.5. In some embodiments, the systems, media and/or methods have a hazard ratio (HR) of greater than about 3.6. In some embodiments, the systems, media and/or methods have a hazard ratio (HR) of greater than about 3.65. In some embodiments, the systems, media and/or methods have a hazard ratio (HR) of at least about 3.68. In some embodiments, the systems, media and/or methods have a hazard ratio (HR) of at least about 4.40. In some embodiments, the systems, media and/or methods have a hazard ratio (HR) of at least about 4.45. In some embodiments, the systems, media and/or methods have a hazard ratio (HR) of at least about 4.50. In some embodiments, the systems, media and/or methods have a hazard ratio (HR) of at least about 4.55. In some embodiments, the systems, media and/or methods have a hazard ratio (HR) of at least about 4.60. In some embodiments, the systems, media and/or methods have a hazard ratio (HR) of between about 3.45 to about 4.80. In some embodiments, the systems, media and/or methods have a hazard ratio (HR) of between about 3.55 to about 4.70.

In some embodiments, the hazard ratio of the dynamic classifier is at least about 5%, 10%, 15%, 20%, 25%, 30%, 35%, 40%, 45%, 50%, 55%, 60%, 65%, 70%, 75%, 80%, 85%, or 90% greater than the hazard ratio of a static predictor. In some embodiments, the hazard ratio of the dynamic classifier at least about 5% greater than the hazard ratio of a static predictor. In some embodiments, the hazard ratio of the dynamic classifier at least about 25% greater than the hazard ratio of a static predictor. In some embodiments, the hazard ratio of the dynamic classifier at least about 50% greater than the hazard ratio of a static predictor. In some embodiments, the hazard ratio of the dynamic classifier at least about 60% greater than the hazard ratio of a static predictor.

In some embodiments, the sensitivity of the systems, media and methods of diagnosing, predicting, or monitoring a status or outcome of a cancer in a subject in need thereof is at least about 0.50, 0.55, 0.60, 0.65, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.90. In some embodiments, the sensitivity is at least about 0.75. In some embodiments, the sensitivity is at least about 0.80. In some embodiments, the sensitivity is at least about 0.84. In some embodiments, the sensitivity of the dynamic classifier is greater than the specificity of a static predictor.

In some embodiments, the specificity of the systems, media and methods of diagnosing, predicting, or monitoring a status or outcome of a cancer in a subject in need thereof is at least about 0.40, 0.45, 0.50, 0.51, 0.52, 0.53, 0.54, 0.55, 0.56, 0.57, 0.58, 0.59, 0.60, 0.65, 0.70, 0.75, 0.80 or 0.90. In some embodiments, the specificity is at least about 0.48. In some embodiments, the specificity is at least about 0.52. In some embodiments, the specificity is at least about 0.55. In some embodiments, the specificity is at least about 0.58. In some embodiments, the specificity of the dynamic classifier is greater than the specificity of a static predictor.

In some embodiments, the accuracy of the systems, media and methods of diagnosing, predicting, or monitoring a status or outcome of a cancer in a subject in need thereof is at least about 0.40, 0.45, 0.48, 0.50, 0.52, 0.55, 0.57, 0.60, 0.61, 0.62, 0.63, 0.64, 0.65, 0.66, 0.67, 0.68, 0.69, 0.70, 0.72, 0.74, 0.76, 0.78, 0.80 or 0.84. In some embodiments, the accuracy is at least about 0.58. In some embodiments, the accuracy is at least about 0.65. In some embodiments, the accuracy is at least about 0.68. In some embodiments, the accuracy of the dynamic classifier is greater than the accuracy of a static predictor.

In some embodiments, the sensitivity, specificity and/or accuracy of the dynamic classifier is greater than the sensitivity, specificity, and/or accuracy of one or more static predictors. In some embodiments, specificity and accuracy of the dynamic classifier is greater than the specificity and accuracy of one or more static predictors.

### Cancer

In some embodiments, the systems, media and methods disclosed herein are used to analyze a cancer in a subject in need thereof. In some embodiments, the cancer is a malignant tissue, benign tissue, or a mixture thereof. In some embodiments, the cancer is a recurrent and/or refractory cancer. Examples of cancers include, but are not limited to, sarcomas, carcinomas, lymphomas or leukemias.

In some embodiments, the cancer is a sarcoma. In some embodiments, sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

In some embodiments, the cancer is a carcinoma. In some embodiments, carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penile cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. The cancer may be a skin cancer, such as a basal cell carcinoma, squamous, melanoma, nonmelanoma, or actinic (solar) keratosis.

In some embodiments, the cancer is a breast cancer. In some embodiments, the breast cancer is a ductal carcinoma. In some embodiments, the breast cancer is a lobular carcinoma. In some embodiments, the breast cancer is a Stage 0 breast cancer. In some embodiments, the breast cancer is a Stage 1 breast cancer. In some embodiments, the breast cancer is a Stage 2 breast cancer. In some embodiments, the breast cancer is a Stage 3 breast cancer. In some embodiments, the breast cancer is a Stage 4 breast cancer. In some embodiments, the breast cancer is an estrogen receptor (ER)-positive, ER-negative, progesterone (PR)-positive, PR-negative, HER2-positive and/or HER2-negative breast cancer. In some embodiments, the breast cancer is a triple-negative breast cancer. In some embodiments, the triple-negative breast cancer is ER-negative, PR-negative and HER2-negative.

In some embodiments, the cancer is a lung cancer. In some embodiments, lung cancer starts in the airways that branch off the trachea to supply the lungs (bronchi) or the small air sacs of the lung (the alveoli). Lung cancers include, but are not limited to, non-small cell lung carcinoma (NSCLC), small cell lung carcinoma, and mesotheliomia. Examples of NSCLC include squamous cell carcinoma, adenocarcinoma, and large cell carcinoma. In some embodiments, the mesothelioma is a cancerous tumor of the lining of the lung and chest cavity (pleura) or lining of the abdomen (peritoneum). In some embodiments, the mesothelioma is due to asbestos exposure. In some embodiments, the cancer is a brain cancer, such as a glioblastoma.

Alternatively, the cancer is a central nervous system (CNS) tumor. In some embodiments, CNS tumors are classified as gliomas or nongliomas. In some embodiments, the glioma is a malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some embodiments, the cancer is a meningioma.

In some embodiments, the cancer is a leukemia. In some embodiments, the leukemia is an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic leukemia.

In some embodiments, the cancer is a lymphoma. In some embodiments, lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

In some embodiments, the systems, media and methods disclosed herein comprise data input from a plurality of cancer cases. In some embodiments, the plurality of cancer cases comprise at least about 10, 20, 30, 40, 50, 60, 70, 80, 90, 100 or more cancer cases. In some embodiments, the plurality of cancer cases comprise at least about 100, 200, 300, 400, 500, 600, 700, 800, 900, 1000 or more cancer cases. In some embodiments, the plurality of cancer cases comprise at least about 1000, 2000, 3000, 4000, 5000, 6000, 7000, 8000, 9000, 10000 or more cancer cases. In some embodiments, the plurality of cancer cases comprise at least about 1000 cancer cases. In some embodiments, the plurality of cancer cases comprise at least about 2000 cancer cases. In some embodiments, the plurality of cancer cases comprise at least about 3000 cancer cases.

In some embodiments, the systems, media and methods disclosed herein comprise data input comprising gene expression profiles for 1 or more genes. In some embodiments, the data input comprise a gene expression profile for at least about 5, 10, 15, 20, 25, 30, 35, 40, 45, 50, 55, 60, 65, 70, 75, 80, 85, 90, 95, 100 or more genes. In some embodiments, the data input comprise a gene expression profile for at least about 100, 200, 300, 400, 500, 600, 700, 800, 900, 1000 or more genes. In some embodiments, the data input comprise a gene expression profile for at least about 1000, 2000, 3000, 4000, 5000, 6000, 7000, 8000, 9000, 10000 or more genes. In some embodiments, the data input comprise a gene expression profile for at least about 25 genes. In some embodiments, the data input comprise a gene expression profile for at least about 100 genes. In some embodiments, the data input comprise a gene expression profile for at least about 500 genes. In some embodiments, the data input comprise a gene expression profile for at least about 750 genes.

### Samples

In some embodiments, the data from the subject suffering from a cancer is based on analysis of one or more samples from the subject suffering from a cancer. In some embodiments, the samples from a cell, tissue, organ, biopsy, fine needle aspirate, bodily fluid, or a combination thereof. In some embodiments, the organ is an adrenal glands, anus, appendix, bladder, bones, brain, bronchi, ears, esophagus, eyes, gall bladder, genitals, heart, hypothalamus, kidney, kidneys, larynx (voice box), liver, lungs, large intestine, lymph nodes, meninges, mouth, nose, pancreas, parathyroid glands, pituitary gland, rectum, salivary glands, skin, skeletal muscles, small intestine, spinal cord, spleen, stomach, thymus gland, thyroid, tongue, trachea, ureters, urethra, or a combination thereof. In some embodiments, the bodily fluid is secreted or excreted. Examples of bodily fluids include, but are not limited to, blood, serum, plasma, sweat, tears, urine, saliva, pus, cerebrospinal fluid, earwax, feces, bile, vaginal secretions, gastric acid, gastric juice, mucus, pericardial fluid, peritoneal fluid, pleural fluid, rheum, sebum, semen, sputum, synovial fluid, and vomit.

### Therapeutic Regimens

In some embodiments, the systems, media and methods disclosed herein comprise predicting a response to a therapeutic regimen. In other instances, the systems, media and methods disclosed herein comprise administering or modifying a therapeutic regime. In some instances, the therapeutic regimen comprises one or more anticancer therapies. Examples of anti-cancer therapies include surgery, chemotherapy, radiation therapy, immunotherapy/biological therapy, photodynamic therapy, or a combination thereof.

In some embodiments, the therapeutic regimen comprises surgery. Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or MRI may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

In some embodiments, the therapeutic regimen comprises one or more chemotherapeutic agents. Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

In some embodiments, the therapeutic regimen comprises one or more anti-metabolites. Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

In some embodiments, the therapeutic regimen comprises one or more alkaloids. Alkaloids may be derived from plants, block cell division, and may also be used for the treatment of cancer. Alkaloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

In some embodiments, the therapeutic regimen comprises one or more podophyllotoxins and/or warfarin (coumadin, dicoumarol). Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase). Warfarin is a synthetic derivative of dicoumarol, a 4-hydroxycoumarin-derived mycotoxin anticoagulant.

In some embodiments, the therapeutic regimen comprises one or more topoisomerases. Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide. Alternatively, the anti-cancer agent comprises a proteasome inhibitor. Examples of proteasome inhibitors include bortezomib, disulfuram, epigallocatechin-3-gallage, salinosporamide A, carfilzomib, ONX912, CEP-18770, and MLN9708.

In some embodiments, the therapeutic regimen comprises one or more cytotoxic antibiotics. Cytotoxic antibiotics are a group of antibiotics that are used for the treatment of cancer because they may interfere with DNA replication and/or protein synthesis. Cytotoxic antibiotics include, but are not limited to, actinomycin, anthracyclines, doxorubicin, daunorubicin, valrubicin, idarubicin, epirubicin, bleomycin, plicamycin, and mitomycin.

In some embodiments, the therapeutic regimen comprises radiation therapy. In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

In some embodiments, the therapeutic regimen comprises external-beam radiation therapy. External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

In some embodiments, the therapeutic regimen comprises intensity-modulated radiation therapy (IMRT). Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose will be delivered from each of the planned beams. The goal of IMRT is to increase the radiation dose to the areas that need it and reduce radiation exposure to specific sensitive areas of surrounding normal tissue.

In some embodiments, the therapeutic regimen comprises image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

In some embodiments, the therapeutic regimen comprises tomotherapy. Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

In some embodiments, the therapeutic regimen comprises stereotactic radiosurgery. Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

In some embodiments, the therapeutic regimen comprises stereotactic body radiation therapy (SBRT). Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In some embodiments, the therapeutic regimen comprises proton therapy. In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

In some embodiments, the therapeutic regimen comprises charged particle beams. Other charged particle beams such as electron beams may be used to irradiate superficial tumors, such as skin cancer or tumors near the surface of the body, but they cannot travel very far through tissue.

In some embodiments, the therapeutic regimen comprises internal radiation therapy. Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a prostate tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

In some embodiments, the therapeutic regimen comprises low-dose-rate or a high-dose-rate radiation treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Brachytherapy may be used to treat patients with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In some embodiments, the therapeutic regimen comprises systemic radiation therapy. In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

In some embodiments, the therapeutic regimen comprises biological therapy. Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

In some embodiments, the therapeutic regimen comprises one or more interferons. Interferons (IFNs) are types of cytokines that occur naturally in the body. Interferon alpha, interferon beta, and interferon gamma are examples of interferons that may be used in cancer treatment.

In some embodiments, the therapeutic regimen comprises one or more interleukins Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

In some embodiments, the therapeutic regimen comprises one or more colony-stimulating factors (CSFs). Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

In some embodiments, the therapeutic regimen comprises monoclonal antibodies (MOABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

In some embodiments, the therapeutic regimen comprises one or more cancer vaccines. Cancer vaccines are another form of biological therapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

In some embodiments, the therapeutic regimen comprises gene therapy. Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some embodiments, the therapeutic regimen comprises one or more nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

In some embodiments, the therapeutic regimen comprises photodynmaic therapy (PDT). Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

In some embodiments, the therapeutic regimen comprises extracorporeal photopheresis (ECP). Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

In some embodiments, the therapeutic regimen comprises laser therapy. Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO2) lasers, argon lasers, and neodymium:yttrium-aluminum-garnet (Nd:YAG) lasers. Each of these can shrink or destroy tumors and can be used with endoscopes. CO2 and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

### EXAMPLES

### Example 1

### Database Construction

In order to establish the largest possible pool of potential training cases for predictor building, we assembled all publicly available breast cancer gene expression data sets that had survival and treatment annotation. We searched the GEO database (http://www.ncbi.nlm.nih.gov/geo/) using the keywords “breast”, “cancer”, “microarray”, and “affymetrix”. Only publications with raw gene expression data, clinical survival information, and at least 30 patients were included. We identified a total of 6,197 cases in 25 datasets.

We further restricted our search to data generated on the HG-U133A (GPL6) and HG-U133 Plus 2.0 (GPL570) arrays only to minimize difficulties of predictor building across different platforms.

We performed a quality check for all arrays and included only arrays with background between 19 and 218, raw Q between 0.5-14, percent present calls >30%, GAPDH 3′:5′ ratio<4.3, beta-actin 3′:5′ ratio<18 and the presence of bioB-/C-/D-spikes as described previously (B. Gyorffy, Z. Benke, A. Lanczky et al., Breast Cancer Res Treat 132 (3), 1025 (2012)).

We also removed duplicate samples (n=1,418)—when multiple GEO entries for the same case existed we retained the first published copy of an array (B. Gyorffy and R. Schafer, Breast Cancer Res Treat 118 (3), 433 (2009)). The final number of unique cases that passed the above QC filters and were included in our master data base was n=3,999. Of these cases, 3,534 had relapse-free survival information (Table 1.).

TABLE 1
Clinical characteristics of patients included in the pooled datasets

| | | | HER2 status | | | | | | | |
|---|---|---|---|---|---|---|---|---|---|---|
| | | | HER2− | | | | | | HER2+ | |
| | | | ER status | | | | | | | |
| | | | ER+ | | | | ER− | | (ER+ and ER−) | |
| | | | Adjuvant therapy | | | | | | | |
| | All | | No systemic | | Adjuvant | | Adjuvant | | Adjuvant | |
| | patients | | therapy | | therapy | | therapy | | therapy | |
| n | 3,534 | | 672 | | 1,316 | | 427 | | 551 | |
| ER+ | 2,960/3,534 | (83.1%) | (all) | | (all) | | (none) | | 372/551 | (66.8%) |
| LN+ | 992//3,220 | (30.8%) | 3/672 | (.4%) | 564/1,083 | (52.1%) | 195/324 | (60.2%) | 147/465 | (31.6%) |
| Grade 1 | 329/2,185 | (15.6%) | 143/528 | (27.0%) | 132/815 | (16.1%) | 10/326 | (3.1%) | 17/291 | (5.8%) |
| Grade 2 | 842/2,185 | (38.5%) | 306/528 | (58.0%) | 355/815 | (43.6%) | 45/326 | (13.8%) | 97/291 | (33.3%) |
| Grade 3 | 964/2,185 | (44.1%) | 78/528 | (14.8%) | 297/815 | (36.5%) | 271/326 | (83.1%) | 177/291 | (60.8%) |

| | | | | | |
|---|---|---|---|---|---|
| Recurrence | 11.60/3,534 | 229/672 | 357/1,316 | 107/427 | 237/551 |
| events | | | | | |
| Median RFS | 5.85 | 7.85 | 5.42 | 3.44 | 5.51 |
| (years) | | | | | |
| Median age | 53.2 | 55.2 | 55.5 | 49.9 | 51.5 |
| (year) | | | | | |
| Median size | 2.3 | 2.0 | 2.49 | 2.0 | 2.35 |
| (cm) | | | | | |

The raw .CEL files were MAS5 normalized in the R statistical environment (http://www.r-project.org) using the affy Bioconductor library (L. Gautier, L. Cope, B. M. Bolstad et al., Bioinformatics 20 (3), 307 (2004)). MASS was used because it performed among the best normalization methods compared to RT-PCR measurements in our previous study (B. Gyorffy, B. Molnar, H. Lage et al., PLoS One 4 (5), e5645 (2009)).

For predictor building only probe sets that were measured by both the GPL96 and GPL570 arrays (n=22,277) were used. We also performed a second scaling normalization to set the average expression of each array to 1000 to reduce batch effects, and subsequently applied an intensity and frequency filter.

Only probe sets for which at least one of the 3,534 samples showed a normalized expression value of 1000 were retained for predictor building. For genes targeted by multiple probe sets only the JetSet best probe16 was retained. The final number of probe sets/genes included in the training database pool for each case was n=9,886.

### Example 2

### Selection of Case-Specific Training Subset and Predictor Building

To select samples for model building (i.e. training subset) we identified cases that were most similar to the test case by computing Euclidean distance with the “dist” function in R to yield a global similarity matrix over all genes. This distance is computed between the test case and each of the samples in the database. We ranked cases by this similarity metric and to study the effect of training set size on predictor performance we built predictors from the top 100, 200, 300, 400 and 500 cases most similar to the test case.

Informative genes were selected for predictor model building by performing a Kaplan-Meier survival analysis for each gene using the median expression values as a cutoff (B. Gyorffy, A. Lanczky, A. C. Eklund et al., Breast Cancer Res Treat 123 (3), 725 (2010)). Genes were ranked by p value and hazard ratio and the average expression of the top 3, 5, 10, 25, 50, 100 and 200 genes were used to make a prognostic prediction. Since some genes correlate positively with survival and have higher expression values in the good prognosis group while others show the opposite relationship, for each gene the difference to the median in the training set is used. In case the hazard ratio is <1, the expression value is inverted to a negative value.

The same processing steps are performed for the test case. The average expression of the informative genes in the test case is compared to the median of the average expression of these genes in the good and the poor outcome groups in the training set (e.g. “molecular classification”).

Adjustment for Clinical Risk

Since selection of the training set cases is driven by molecular similarities to the test case, the resulting training cohort could have unbalanced clinical features that could skew overall prognostic prediction. For example, if the training cohort includes a large number of cases with poor clinical risk features (i.e. mostly node-positive, mostly high grade, large cancers, etc. . . . ), the overall prognostic risk prediction based on molecular features alone may be erroneous. For this reason, the entire training set is compared to all the remaining patients using a Kaplan-Meier analysis. The results of this analysis termed “training set assessment” are used in the final prognostic classification to adjust the molecular risk that is based on molecular features alone.

The Final Classification Rule

The final classification rule takes into account both the risk assignment from the “training set assessment” and the output from the “molecular classification”. When both predictors are concordant and assign good or poor prognosis, the decision rule follows the concordant vote.

When the “molecular classification” is not significant for either good or poor prognosis or when the clinical prediction contradicts the molecular prediction the final output is “intermediate”.

### Example 3

### Optimization of Training Set Size and Informative Gene Set Size

To measure the performance of our dynamic classifier, we performed a leave-one out cross validation (LOOCV) for each 3,534 samples (e.g. one case held out and the training subset selected from the remaining 3,533). We first examined the influence of training set size and the number of informative genes included in the predictor on the performance of our classification method. The LOOCV was performed for a range of these parameters including genes from 3 to 200 and training cohorts from 100 to 500. To estimate performance differences between predictors, all of the chi-square results of the logrank test comparing the survival curves generated by different predictors were compared by a paired t-test.

### Example 4

### Construction of Online Interface

To enable the classification of new samples by any user, we developed an online interface. In this, all computations on the microarray data are performed in real time on a Debian linux (http://www.debian.org) central server. This server runs an Apache webserver, a (D)COM server, and a background R server. After the upload of the .CEL file, the data is loaded into the R environment, where QC and normalization are performed. The packages “affy” and “survival” are used for normalization and for drawing Kaplan-Meier plots, respectively. The homepage was set up using a modular and open source Drupal content management system (http://www.drupal.org). The results are provided at the end of the analysis directly on the webpage. The homepage can be accessed at http://www.recurrenceonline.com/?q=Re_training.

### Example 5

### Computation of Static Predictors

We compared the overall performance of our optimized dynamic predictors (using case-specific training set of 400 with top 25 most informative genes) to genomic surrogates of three commonly used static predictors, the 21-gene recurrence score, the 70-gene Mammaprint signature classifier and the 97-gene genomic grade index (GGI). For computing the recurrence score, we used our previously published technique12. The GGI and the 70-gene classifications were computed using the “genefu” Bioconductor package (http://www.bioconductor.org) using the default parameters.

We computed sensitivity as =TP/(TP+FN) where TP=number of true positives, and FN=number of false negatives; specificity as =TN/(TN+FP) where TN=number of true negatives and FP=number of false positives and accuracy as =(TP+TN)/(TP+FN+TN+FP). In the analysis, the predictive power of relapse up to 5 years was compared. Patients censored before 5 years were excluded from the analysis (final n=2,801).

The dynamic re-training algorithm was applied to each sample as well as the three genomic surrogated described herein. The performance of the classifiers was assessed by computing Cox regression and plotting a Kaplan-Meier plot for each classification algorithm separately.

Independent Validation Samples

We obtained 325 independent validation samples of early stage breast cancers from collaborators at the Departments of Gynecology and Obstetrics at the University Hospitals in Frankfurt and Hamburg, Germany. All patients participated in an IRB approved study and signed informed consent for biomarker analysis. They represent consecutive patients undergoing surgical resection up to July 2007. The median age of was 56 years, 81% of the cancers were ER positive, 40% were lymph node positive, 60% were >2 cm in size, and 32% was high histological grade (G3). Thirty seven percent of patients received adjuvant endocrine therapy and 63% received adjuvant chemotherapy. Samples were annotated with standard pathology including ER status by ligand binding assays or immunohistochemistry. All tissue samples were stored in liquid nitrogen until gene expression profiling. Isolation of RNA and expression profiling using Affymetrix Human Genome U133A microarrays was performed according to the manufacturer's protocols. Affymetrix data and CEL files have been deposited in the GEO database

### Example 6

### Optimization of Predictor Parameters for Dynamic Prediction

First we examined the impact of varying the training set size and number of informative genes included in the predictor on predictor performance. Predictor parameter optimization was done by leave one out cross validation for 3,534 patients by varying the number of genes included in the predictor (3-200) and the size of training subsets (100-500). The average Chi-square values, hazard ratios and p values are shown. The chi-square results are color coded; from green to red the colors correspond to increasing better prognostic discrimination. The highest classification efficiency was achieved by using 25 genes and including 400 samples in the training set. The resulting average chi-square, hazard ratio and p-values comparing the survival in the good and bad prognosis groups for each analysis are summarized in Table 2.

TABLE 2

| training | | Number of genes used for classification | | | | | | | |
|---|---|---|---|---|---|---|---|---|---|
| set size | | 3 | 5 | 10 | 25 | 50 | 100 | 200 | average |
| 100 | chi2 | 236 | 232 | 226 | 231 | 287 | 303 | 283 | 257 |
| | p-value | 4.66E−52 | 3.33E−51 | 9.34E−50 | 5.53E−51 | 4.26E−63 | 1.75E−66 | 3.67E−62 | 1.47E−50 |
| | HR | 2.55 | 2.45 | 2.48 | 2.54 | 3.11 | 3.07 | 3.05 | 2.75 |
| 200 | chi2 | 284 | 287 | 306 | 298 | 297 | 294 | 315 | 297 |
| | p-value | 2.30E−62 | 4.18E−63 | 4.48E−67 | 1.58E−65 | 2.80E−65 | 1.29E−64 | 4.57E−69 | 3.9E−63 |
| | HR | 3.03 | 2.98 | 3.18 | 3.18 | 3.36 | 3.45 | 3.68 | 3.265714 |
| 300 | chi2 | 281 | 284 | 300 | 288 | 292 | 269 | 293 | 287 |
| | p-value | 1.01E−61 | 2.57E−62 | 8.89E−66 | 2.98E−63 | 3.57E−64 | 3.94E−59 | 2.23E−64 | 5.65E−60 |
| | HR | 2.9 | 2.9 | 2.91 | 3.12 | 3.35 | 3.13 | 3.48 | 3.112857 |
| 400 | chi2 | 317 | 303 | 322 | 325 | 310 | 294 | 306 | 311 |
| | p-value | 1.77E−69 | 1.84E−66 | 1.54E−70 | 2.20E−71 | 5.78E−68 | 1.63E64 | 4.53E−67 | 2.37E−65 |
| | HR | 3.17 | 3.19 | 3.52 | 3.68 | 3.51 | 3.54 | 3.6 | 3.458571 |
| 500 | chi2 | 302 | 297 | 302 | 313 | 302 | 288 | 290 | 299 |
| | p-value | 2.47E−66 | 3.69E−65 | 2.46E−66 | 1.14E−68 | 2.13E−66 | 2.93E−63 | 9.97E−64 | 5.68E−64 |
| | HR | 3.18 | 3.15 | 3.24 | 3.74 | 3.73 | 3.56 | 3.7 | 3.471429 |
| average | chi2 | 284 | 281 | 291 | 291 | 298 | 290 | 297 | |
| | p-value | 9.32E−53 | 6.66E−52 | 1.87E−50 | 1.11E51 | 9.3E−64 | 7.89E−60 | 7.59E−63 | |
| | HR | 2.966 | 2.934 | 3.066 | 3.252 | 3.412 | 3.35 | 3.502 | |

We examined the effect of increasing training set size from 100 to 500 patients in increments of 100. The corresponding average chi-square values (of all performed analyses across all tested gene set sizes) were 257, 297, 287, 311 and 299, respectively. The improvement in chi-square values was significant up until training set size of 400 (t-test of chi-square distributions, p=0.0005), but it significantly deteriorated when sample size was extended to 500 cases (p=0.024). Because of this deterioration in performance and because of the substantially increasing computational time when including >500 patients in the training set we have not tested larger training set sizes.

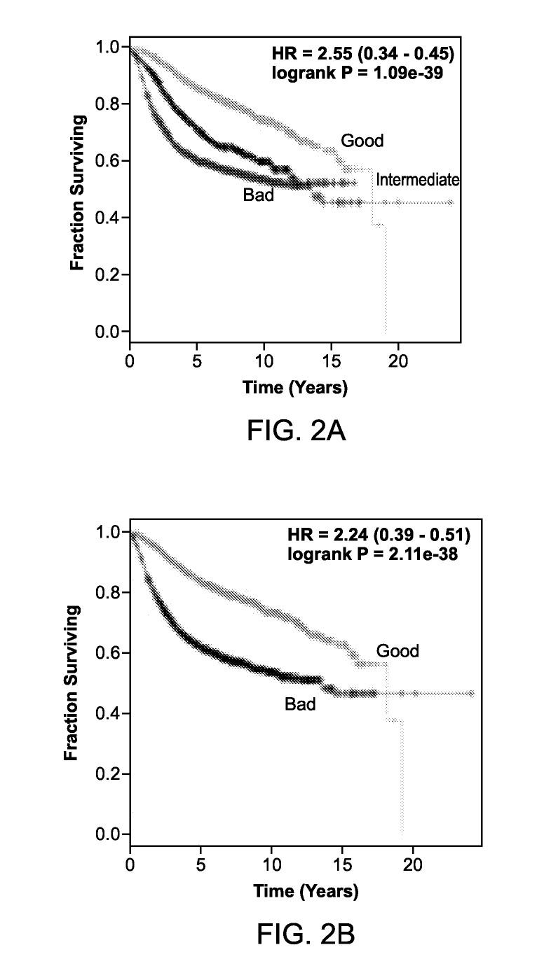

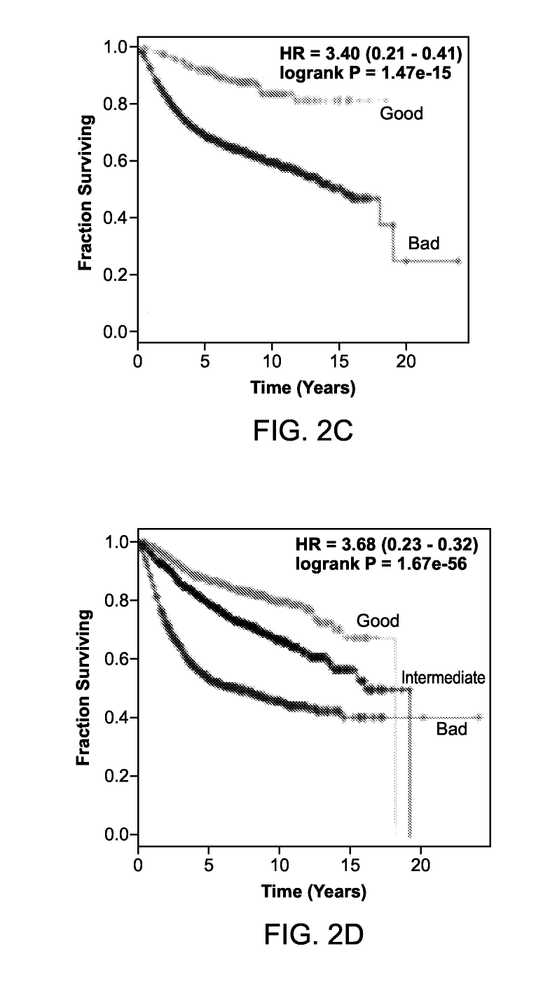

The informative gene set size was also varied including 3, 5, 10, 25, 50, 100 and 200 genes. The corresponding average chi-square values across all training set sizes were 284, 281, 291, 298, 290 and 297, respectively. Although these differences were not significant, the nominally best classification was be achieved by the combination of 25 genes and 400-patient training set. The Kaplan-Meier survival plot for this optimized predictor calculated over all cases is presented in FIG. 2A-D.

### Example 7

### Comparison of the Dynamic Predictor to Previously Published Static Classifiers

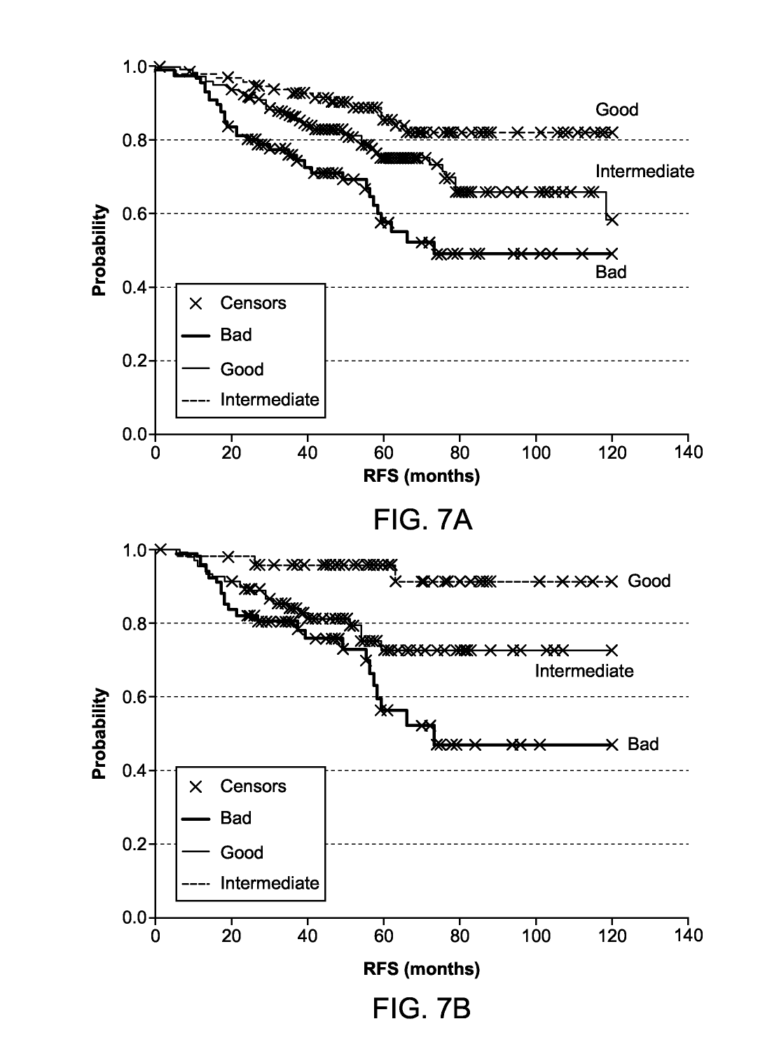

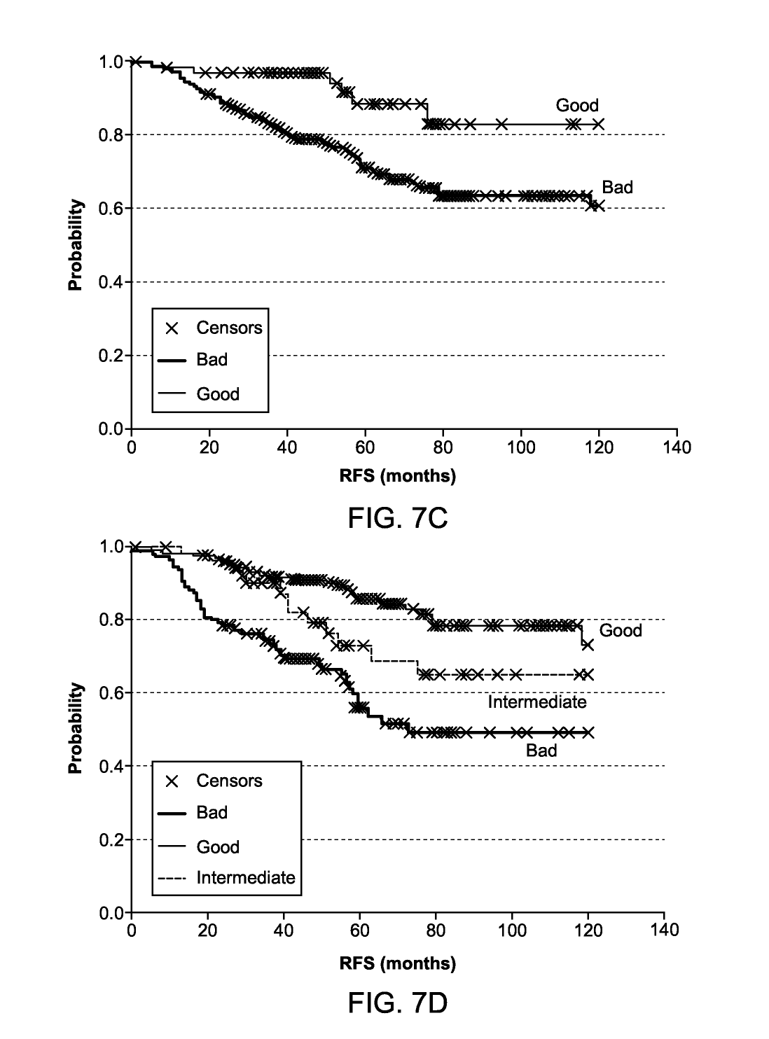

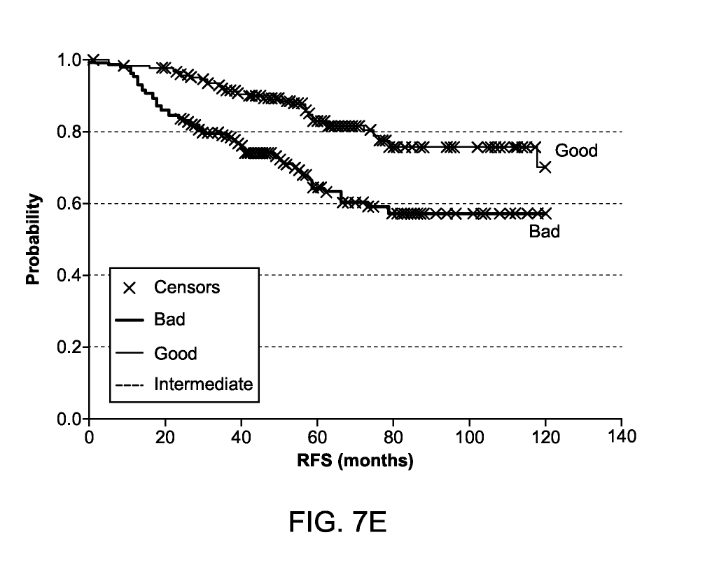

We applied commonly used genomic surrogates of the 21-gene recurrence score, the 70-gene prognostic signature and the 97-gene genomic grade index to our entire data set (FIG. 7).

For all patients, the dynamic prediction method yielded the highest hazard ratio (HR=3.68) followed by the 70-gene classifier (HR=3.40), the 21-gene recurrence score (HR=2.55) and the 97-gene genomic grade index (HR=2.24) (FIG. 2A-D).

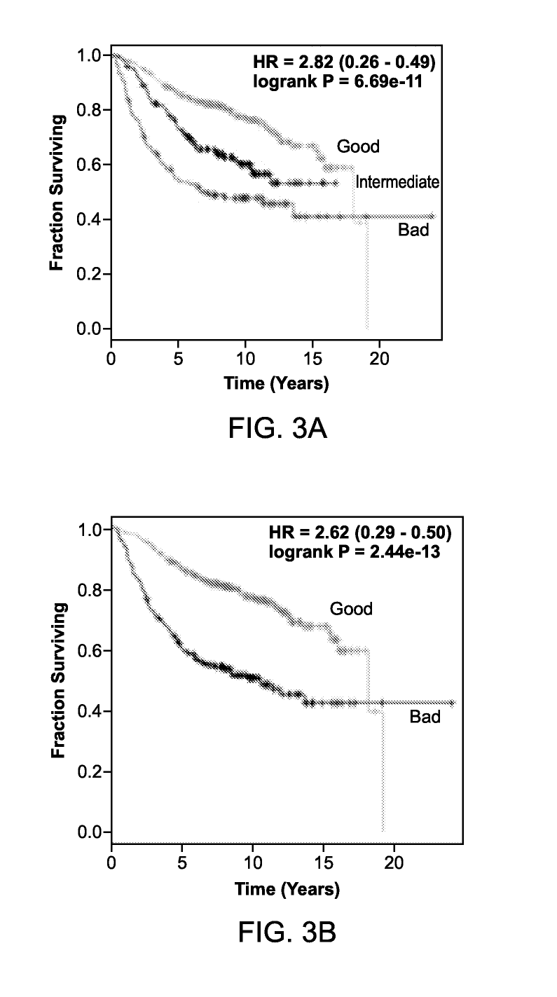

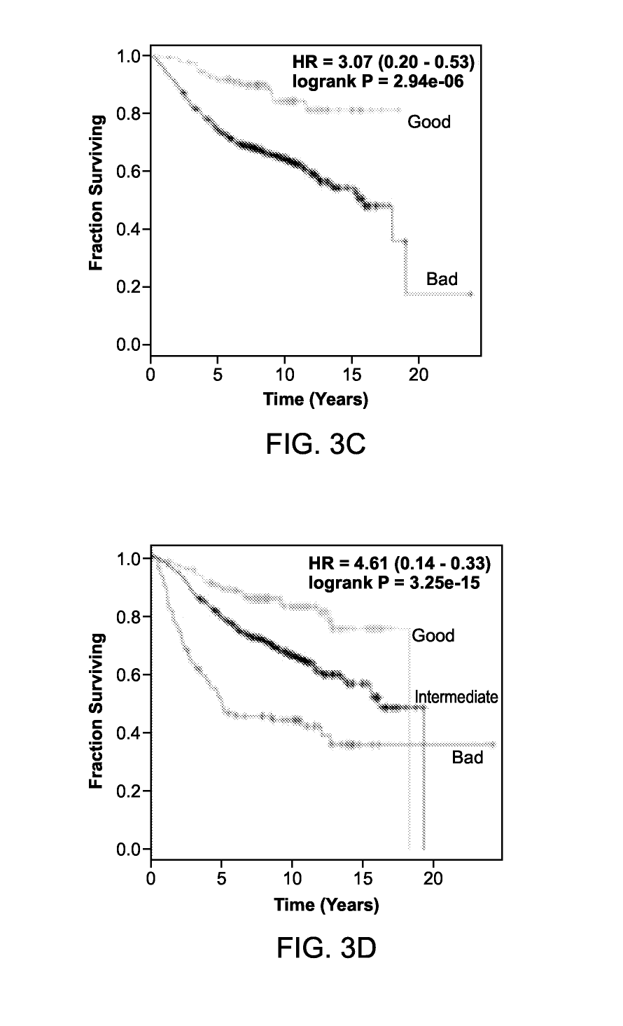

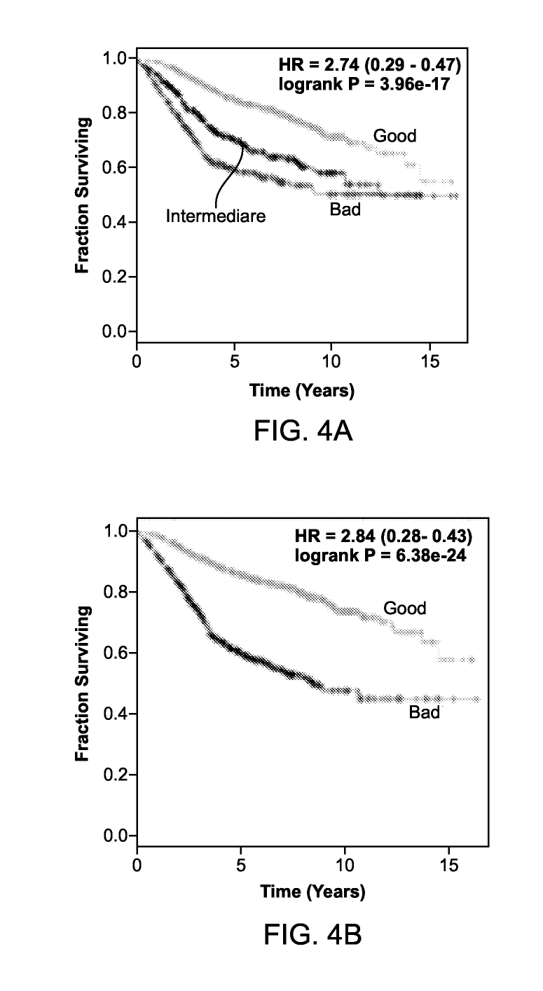

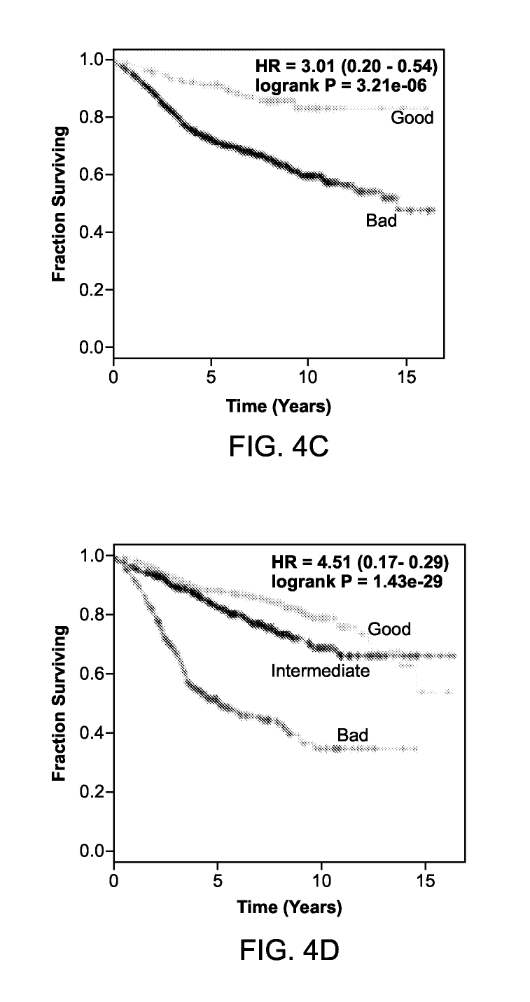

This also remained true for ER positive/HER2 negative patients without adjuvant chemotherapy (dynamic predictor HR=4.61, 70-gene HR=3.07, 21-gene HR=2.82, 97-gene HR=2.62) (FIG. 3A-D). The dynamic predictor also performed best for ER positive/HER2 negative patients who received adjuvant chemotherapy (dynamic predictor HR=4.51, 70-gene HR=3.01, 97-gene HR=2.84, 21-gene HR=2.74) (FIG. 4A-D).

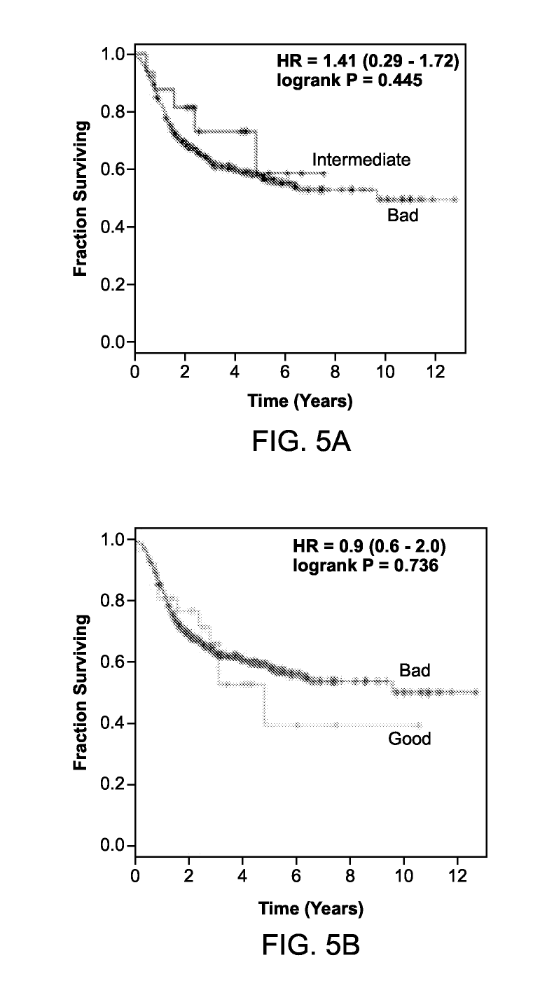

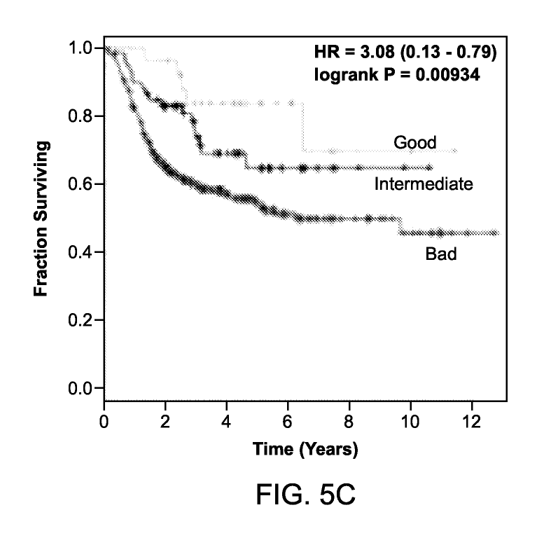

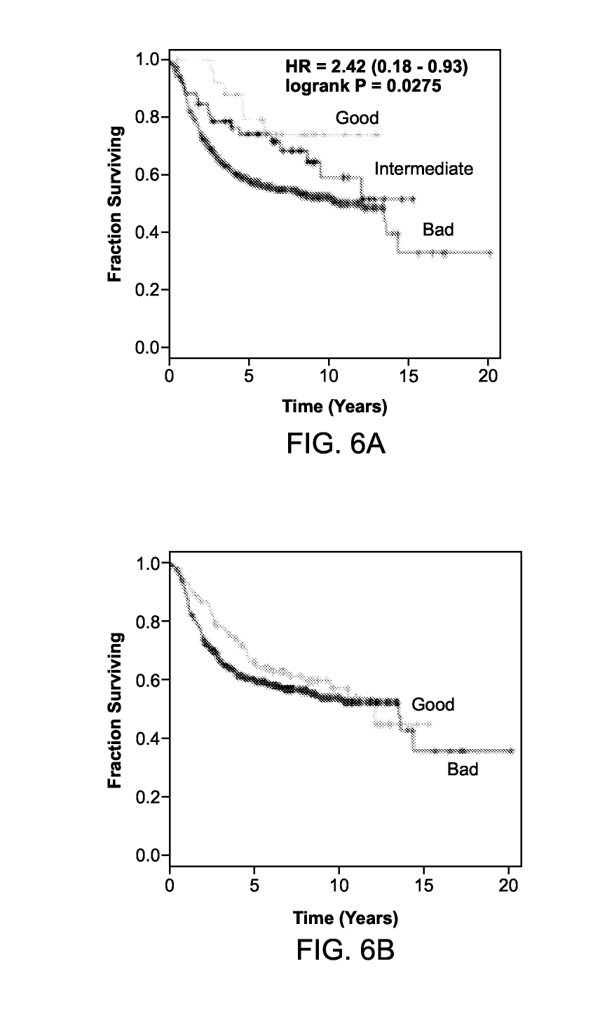

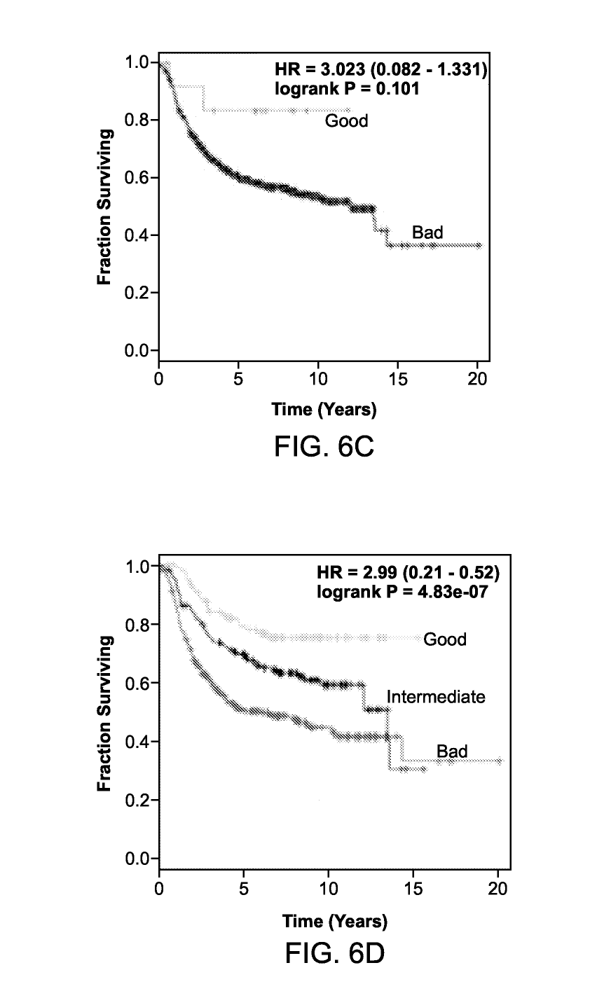

Most importantly, for ER negative/HER2 negative patients only the dynamic predictor achieved significant discriminating power (HR=3.08, p=0.009) (FIG. 5A-C). The 97-gene GGI and the 21-5 gene recurrence score delivered a classification in these cohorts, but failed to achieve significance. In HER2 positive patients (including both ER positive and negative cases), only the dynamic classification method (HR=2.99) and the 21-gene recurrence score (HR=2.42) were capable to achieve significance (FIG. 6A-D).

We also assessed the sensitivity, specificity and accuracy of each method for predicting relapse-free survival at five years. The highest sensitivity was achieved by the 70-gene signature (0.98) but it had the lowest specificity (0.13). This predictor also assigned a large proportion of patients to high risk category. The dynamic predictor had sensitivity of 0.84, the 21-gene signature 0.80 and the 97-gene signature 0.75. The highest specificity (0.58) was achieved by the dynamic classifier, followed by the 21-gene score (0.55), the 97-gene signature (0.45) and the 70-gene signature. The dynamic classification method also had the highest overall accuracy (0.68), followed by the 21-gene score (0.64), the 97-gene signature (0.55) and the 70-gene signature (0.41) (see Table 3).

TABLE 3
Performance comparison of the different predictors
for overall sensitivity, specificity and accuracy.

| | Dynamic | | | |
|---|---|---|---|---|
| | reclassi- | 21-gene | 70-gene | 97-gene |
| | fication | signature | signature | signature |
| Sensitivity | 0.84 | 0.80 | 0.98 | 0.75 |
| Specificity | 0.58 | 0.55 | 0.13 | 0.45 |
| Accuracy | 0.68 | 0.64 | 0.41 | 0.55 |

TABLE 4
Comparison of numbers at risk for different predictors corresponding
to all patients (n = 3,534 -corresponding to FIG. 2A-D)

| | | 0 years | 5 years | 10 years | 15 years | 20 years |
|---|---|---|---|---|---|---|
| 21-gene | Bad (High) | 1691 | 673 | 215 | 21 | 2 |
| (Oncotype Dx) | Intermediate | 649 | 354 | 110 | 8 | 0 |
| | Good (Low) | 1194 | 837 | 288 | 40 | 0 |
| GGI | Bad (High) | 2009 | 838 | 257 | 26 | 2 |
| | Good (Low) | 1525 | 1026 | 356 | 43 | 0 |
| 70-gene | Bad (High) | 3236 | 1626 | 528 | 56 | 2 |
| (MammaPrint) | Good (Low) | 298 | 238 | 85 | 13 | 0 |
| Dynamic | Bad (High) | 1400 | 471 | 150 | 17 | 2 |
| Reclassi- | Good (Low) | 915 | 638 | 223 | 20 | 0 |
| fication | Intermediate | 1219 | 755 | 240 | 32 | 0 |

TABLE 5
Comparison of numbers at risk for different predictors corresponding
to ER+ HER2− patients (untreated, n = 672 -corresponding to FIG. 3A-D)

| | | 0 years | 5 years | 10 years | 15 years | 20 years |
|---|---|---|---|---|---|---|
| 21-gene | Bad (High) | 115 | 59 | 32 | 5 | 1 |
| (Oncotype Dx) | Intermediate | 159 | 111 | 43 | 4 | 0 |
| | Good (Low) | 398 | 322 | 153 | 35 | 0 |
| GGI | Bad (High) | 217 | 122 | 53 | 8 | 1 |
| | Good (Low) | 455 | 370 | 175 | 36 | 0 |
| 70-gene | Bad (High) | 549 | 384 | 174 | 32 | 1 |
| (MammaPrint) | Good (Low) | 123 | 108 | 54 | 12 | 0 |
| Dynamic | Bad (High) | 97 | 47 | 28 | 3 | 1 |
| Reclassi- | Good (Low) | 221 | 181 | 82 | 15 | 0 |
| fication | Intermediate | 354 | 264 | 118 | 26 | 0 |

TABLE 6
Comparison of numbers at risk for different predictors
corresponding to ER+ HER2− patients
(treated, n = 1316 -corresponding to FIG. 4A-D)

| | | 0 years | 5 years | 10 years | 15 years |
|---|---|---|---|---|---|
| 21-gene | Bad (High) | 356 | 110 | 28 | 0 |
| (Oncotype Dx) | Intermediate | 331 | 146 | 37 | 3 |
| | Good (Low) | 629 | 382 | 99 | 5 |
| GGI | Bad (High) | 576 | 193 | 43 | 2 |
| | Good (Low) | 740 | 445 | 121 | 6 |
| 70-gene | Bad (High) | 1180 | 540 | 138 | 7 |
| (MammaPrint) | Good (Low) | 136 | 98 | 26 | 1 |
| Dynamic | Bad (High) | 400 | 104 | 18 | 0 |
| Reclassi- | Good (Low) | 404 | 254 | 78 | 4 |
| fication | Intermediate | 512 | 280 | 68 | 4 |

TABLE 7
Comparison of numbers at risk for different predictors corresponding
to ER− HER2− patients (treated. n = 427 -corresponding to FIG. 5A-D)

| | | 0 yrs | 2 yrs | 4 yrs | 6 yrs | 8 yrs | 10 yrs | 12 yrs |
|---|---|---|---|---|---|---|---|---|
| 21-gene | Bad (High) | 411 | 245 | 126 | 54 | 24 | 12 | 1 |
| (Oncotype Dx) | Intermediate | 16 | 13 | 8 | 3 | 0 | 0 | 0 |
| GGI | Bad (High) | 401 | 241 | 127 | 54 | 23 | 11 | 1 |
| | Good (Low) | 26 | 17 | 7 | 3 | 1 | 1 | 0 |
| 70-gene | Bad (High) | Not possible, all high risk | | | | | | |
| (MammaPrint) | Good (Low) | | | | | | | |
| Dynamic | Bad (High) | 341 | 188 | 94 | 40 | 19 | 10 | 1 |
| Reclassi- | Good (Low) | 26 | 25 | 16 | 8 | 2 | 1 | 0 |
| fication | Intermediate | 60 | 45 | 24 | 9 | 3 | 1 | 0 |

TABLE 8
Comparison of numbers at risk for different predictors corresponding to
HER2+ patients (n = 551 -corresponding to FIG. 6A-D)

| | | 0 years | 5 years | 10 years | 15 years | 20 years |
|---|---|---|---|---|---|---|
| 21-gene | Bad (High) | 473 | 217 | 80 | 5 | 1 |
| (Oncotype Dx) | Intermediate | 52 | 34 | 11 | 1 | 0 |
| | Good (Low) | 26 | 18 | 4 | 0 | 0 |
| GGI | Bad (High) | 439 | 206 | 76 | 5 | 1 |
| | Good (Low) | 112 | 63 | 19 | 1 | 0 |
| 70-gene | Bad (High) | 538 | 260 | 94 | 6 | 1 |
| (MammaPrint) | Good (Low) | 13 | 9 | 1 | 0 | 0 |
| Dynamic | Bad (High) | 295 | 108 | 44 | 4 | 1 |
| Classifier | Good (Low) | 98 | 63 | 19 | 1 | 0 |
| | Intermediate | 158 | 98 | 32 | 1 | 0 |

The Most Consistently Strongly Prognostic Genes

To identify the genes with the highest predictive potential, the prevalence of all genes included in the top 25 list from all LOOCV analyses was counted. In the 3,534 runs (one for each case), 5,038 distinct genes were associated with prognosis in at least one case. Of these, only 72 genes were present in more than 5% of classification signatures (n=176).

Web Tool to Provide Dynamic Survival Prediction

We have made the dynamic prognostic classifier available on line. This web-based tool enables users to make prognostic prediction for a new case using our dynamic classification method. It also allows independent validation when it is applied to new data sets. The tool requires uploading unprocessed Affymetrix HGU133A or HGU133plus2 microarray .CEL file in an online interface available at http://www.recurrenceonline.com/?q=Re_training. The entire computational process is performed real time and the result is provided as a Kaplan-Meier survival plot showing the estimated survival of cases with similar molecular and clinical features derived from the pooled data of 3,534 cases.

Validation in Independent Clinical Samples

Three hundred and twenty five cases which are not included in the pooled public data database were used for independent validation of our method. The average follow-up for these patients was 58 months. The dynamic predictor achieved excellent classification efficiency (HR=3.57) and outperformed the 21-gene recurrence score (HR=3.39), the 71-gene signature (HR=3.13) and the GGI signature (HR=2.28). The dynamic predictor remained more effective when applied the chemotherapy treated patients only (n=204, HR=7.72) than to the 21-gene recurrence score (HR=5.97), the 71-gene signature (HR=3.82) or the Genomic Grade Index (HR=3.33). The Kaplan-Meier plots are presented in FIG. 7.

Discussion

High throughput genomic analysis has fundamentally changed our perception of breast cancer and the large scale heterogeneity of this diseases has become widely recognized (B. Weigelt, F. L. Baehner, and J. S. Reis-Filho, J Pathol 220 (2), 263 (2010)). Thus, searching for general prognostic markers that are applicable to all breast cancers is no longer considered appropriate (B. Weigelt, L. Pusztai, A. Ashworth et al., Nat Rev Clin Oncol 9 (1), 58 (2012)). Yet, most currently used prognostic signatures that were developed over a decade ago following the old paradigm of breast cancer as single disease. Here, we present a new approach to prognostic predictor discovery which recognizes heterogeneity of breast cancer and takes advantage of the large number of gene expression data sets that are now available for predictor discovery and training. The main idea of our method is that we define a predictor for a new case from the molecularly most similar cancers. Since each case differ from one another, the predictor and training set also differs from case to case, hence we call our method a dynamic predictor.

We applied our method to gene expression data from 3,534 breast cancers and to a set of 325 independent cases. The dynamic re-training approach yielded higher average classification efficiency then three commonly used first generation prognostic signatures including the 21-gene recurrence score, the 70-gene prognostic signature and the 97-gene genomic grade index. It is important to recognize that our paper compares different conceptual approaches to prognostic prediction rather than results from the actual commercially available prognostic tests.

One of our most important observations is that the dynamic classifier performed substantially better to discriminate between good and poor prognosis among ER-negative/HER negative cancers than any of the first generation gene signatures that we tested. It is well recognized that the prognostic power of the currently clinically available multi-gene prognostic assays is primarily restricted to ER positive cancers and the vast majority of ER negative cancers are assigned to poor prognosis by these tests. Our results suggest that the re-training classification method can also provide prognostic information for triple-negative cancers.

Consistent with previous reports, most of the top ranked genes associated with survival differ from training set to training set. The three most commonly top ranked genes were CENPE, RACGAP1 and PGK1 which were included in the top 25 list in 831, 759 and 756 analyses, respectively. Thus, the most common gene reaches only a prevalence of 23.5% in all analyses. This observation illustrates the instability of gene rankings and also reflects the heterogeneity of breast cancers (C. Curtis, S. P. Shah, S. F. Chin et al., Nature 486 (7403), 346 (2012)).

Our method preserves the independence of model discovery from validation but it does not apply a single fixed predictor to each new test case. A unique, case specific predictor is developed for each new test case. In order to allow other investigators to use and validate our method, we constructed a web-based dynamic prognostic predictor tool that is available at http://www.recurrenceonline.com/?q=Re_training. It requires uploading of an Affymetrix HGU133A or HGU133plus2 microarray .CEL file, then it automatically performs QC assessment and normalization and performs the dynamic risk prediction as described in this paper. This provides a new standardized, low cost, open source paradigm for genomic predictors (C. Sotiriou and L. Pusztai, N Engl J Med 360 (8), 790 (2009)).

To our knowledge, this transcriptome-based algorithm presents the first approach where a dynamic classification tool without a defined gene-list is presented. The ultimate power of the approach lies in the future extension of the database and its applicability to any multivariate predictor which relies on high throughput data and require large training sets from a heterogeneous disease population.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A dynamic computer-implemented method comprising:
receiving, by a computer, data input, the data pertaining to a plurality of breast cancer cases;
generating, by the computer, a case-specific output, wherein the case-specific output comprises a subset of the plurality of breast cancer cases, a subset of the data pertaining to the plurality of breast cancer cases, or a combination thereof, and wherein the case-specific output is based on a comparison of the data pertaining to the plurality of breast cancer cases to data pertaining to a subject suffering from a breast cancer;
generating, by the computer, a biomedical output, the biomedical output comprising a comparison of the data of the case-specific output to the data of the subject suffering from the breast cancer; and
diagnosing, predicting or monitoring, by the computer, a status or outcome of the breast cancer in the subject based on the biomedical output.

2-25. (canceled)

26. The method of claim 1, further comprising ranking two or more breast cancer cases of the plurality of breast cancer cases, wherein the ranking comprises comparing data of the two or more breast cancer cases to the data of the subject so as to determine similarity of the two or more breast cancer cases to the subject.

27-32. (canceled)

33. The method of claim 26, further comprising producing a case-specific training subset based on the ranking of the two or more breast cancer cases, the case-specific training subset comprising a subset of the plurality of breast cancer cases.

34. (canceled)

35. The method of claim 33, wherein the subset of the plurality of breast cancer cases comprises the most similar breast cancer cases to the subject.

36. (canceled)

37. The method of claim 33, wherein the case-specific output comprises the case-specific training subset.

38. The method of claim 33, further comprising ranking two or more genes of one or more breast cancer cases of the case-specific training subset.

39-41. (canceled)

42. The method of claim 38, further comprising producing a case-specific gene set based on the ranking of the two or more genes.

43. The method of claim 42, wherein the case-specific gene set comprises the subset of the data pertaining to the plurality of breast cancer cases, the subset of the data comprising one or more of the highest ranked genes, and wherein the case-specific output comprises the case-specific gene set.

44-46. (canceled)

47. The method of claim 43, wherein the biomedical output comprises one or more molecular classifications based on a comparison of an average expression level of the one or more highest ranked genes of the case-specific output to an average expression level of one or more genes of the subject.

48. (canceled)

49. The method of claim 47, wherein the biomedical output further comprises one or more training set assessments based on a comparison of the case-specific output to one or more additional subjects suffering from breast cancer.

50-63. (canceled)

64. A dynamic computer-implemented system comprising:
a digital processing device comprising an operating system configured to perform executable instructions and a memory device;
a computer program including instructions executable by the digital processing device to create an application comprising:
(i) a software module configured to receive data input, the data pertaining to a plurality of breast cancer cases;
(ii) a software module configured to generate a case-specific output, wherein the case specific output comprises a subset of the plurality of breast cancer cases, a subset of the data pertaining to the plurality of breast cancer cases, or a combination thereof; and
(iii) a software module configured to generate a biomedical output, the biomedical output comprising a comparison of the data of the case-specific output to the data of the subject suffering from the breast cancer.

65-73. (canceled)

74. The system of claim 64, further comprising one or more additional software modules configured to rank two or more breast cancer cases of the plurality of breast cancer cases, wherein the ranking comprises comparing data of the two or more breast cancer cases to the data of the subject so as to determine similarity of the two or more breast cancer cases to the subject.

75-80. (canceled)

81. The system of claim 74, further comprising producing a case-specific training subset based on the ranking of the two or more breast cancer cases, the case-specific training subset comprising a subset of the plurality of breast cancer cases, wherein the case-specific output comprises the case-specific training subset.

82. (canceled)

83. The system of claim 81, wherein the subset of the plurality of breast cancer cases comprises the most similar breast cancer cases to the subject.

84-85. (canceled)

86. The system of claim 81, further comprising one or more additional software modules configured to rank two or more genes of one or more breast cancer cases of the case-specific training subset.

87-89. (canceled)

90. The system of claim 86, further comprising one or more additional software modules configured to generate a case-specific gene set based on the ranking of the two or more genes.

91. The system of claim 90, wherein the case-specific gene set comprises the subset of the data pertaining to the plurality of breast cancer cases, the subset of the data comprising one or more of the highest ranked genes, and wherein the case-specific output comprises the case-specific gene set.

92-94. (canceled)

95. The system of claim 91, wherein the biomedical output comprises one or more molecular classifications based on a comparison of an average expression level of the one or more highest ranked genes of the case-specific output to an average expression level of one or more genes of the subject.

96. (canceled)

97. The system of claim 95, wherein the biomedical output further comprises one or more training set assessments based on a comparison of the case-specific output to one or more additional subjects suffering from a breast cancer.

98-99. (canceled)

100. The system of claim 64, further comprising one or more additional software modules configured to diagnose, predict, or monitor a status or outcome of the breast cancer in the subject based on the biomedical output.

101-121. (canceled)

122. Non-transitory computer-readable storage media encoded with a computer program including instructions executable by a processor to create an application comprising:
a database, in a computer memory, of a plurality of breast cancer cases;
a software module configured to receive data input, the data pertaining to a plurality of breast cancer cases;
a software module configured to generate a case-specific output, wherein the case specific output comprises a subset of the plurality of breast cancer cases, a subset of the data pertaining to the plurality of breast cancer cases, or a combination thereof; and
a software module configured to generate a biomedical output, the biomedical output comprising a comparison of the data of the case-specific output to the data of the subject suffering from the breast cancer.

123-131. (canceled)

132. The storage media of claim 122, further comprising one or more additional software modules configured to rank two or more breast cancer cases of the plurality of breast cancer cases, wherein the ranking comprises comparing data of the two or more breast cancer cases to the data of the subject so as to determine similarity of the two or more breast cancer cases to the subject.

133-138. (canceled)

139. The storage media of claim 132, further comprising producing a case-specific training subset based on the ranking of the two or more breast cancer cases, the case-specific training subset comprising a subset of the plurality of breast cancer cases, wherein the case-specific output comprises the case-specific training subset.

140. (canceled)

141. The storage media of claim 139, wherein the subset of the plurality of breast cancer cases comprises the most similar breast cancer cases to the subject.

142-143. (canceled)

144. The storage media of claim 139, further comprising one or more additional software modules configured to rank two or more genes of one or more breast cancer cases of the case-specific training subset.

145-147. (canceled)

148. The storage media of claim 144, further comprising one or more additional software modules configured to generate a case-specific gene set based on the ranking of the two or more genes.

149. The storage media of claim 148, wherein the case-specific gene set comprises the subset of the data pertaining to the plurality of breast cancer cases, the subset of the data comprising one or more of the highest ranked genes, and wherein the case-specific output comprises the case-specific gene set.

150-152. (canceled)

153. The storage media of claim 149, wherein the biomedical output comprises one or more molecular classifications based on a comparison of an average expression level of the one or more highest ranked genes of the case-specific output to an average expression level of one or more genes of the subject.

154. (canceled)

155. The storage media of claim 153, wherein the biomedical output further comprises one or more training set assessments based on a comparison of the case-specific output to one or more additional subjects suffering from a breast cancer.

156-157. (canceled)

158. The storage media of claim 122, further comprising one or more additional software modules configured to diagnose, predict, or monitor a status or outcome of the breast cancer in the subject based on the biomedical output.

159-179. (canceled)
